(19) United States
(12) Patent Application Publication
(10) Pub. No.: US 2024/0145041 A1
(43) Pub. Date: May 2, 2024

(54) PROCESSES, MACHINES, AND ARTICLES OF MANUFACTURE RELATED TO MACHINE LEARNING FOR PREDICTING BIOACTIVITY OF COMPOUNDS

(71) Applicant: Flagship Pioneering Innovations VI, LLC, Cambridge, MA (US)

(72) Inventors: Hok Hei Tam, Newton, MA (US); Varun Shivashankar, Waltham, MA (US); Nathan Sanders, North Andover, MA (US); Terran Lane, Somerville, MA (US); David Kolesky, Arlington, MA (US); Mostafa Karimi, Redmond, WA (US)

(21) Appl. No.: 18/497,139

(22) Filed: Oct. 30, 2023

Related U.S. Application Data
(63) Continuation of application No. PCT/US22/28336, filed on May 9, 2022.
(60) Provisional application No. 63/186,274, filed on May 10, 2021.

(51) Int. Cl. G16C 20/30; G06N 20/20; G16C 20/50; G16C 20/70

(52) U.S. Cl. CPC G16C 20/30; G06N 20/20; G16C 20/50; G16C 20/70

(57) ABSTRACT

The computer system applies machine learning techniques to train a computational model using data representing researched items and their known properties. The computer system applies the trained computational model to data representing the potential candidate items to predict whether such items have such properties. The trained computational model outputs one or more predictions about whether the potential candidate items are likely to have a property from among the plurality of types of properties that the computational model is trained to predict. The computer system allows multiple machine learning experiments to be defined, and then allows predictions from those multiple machine learning experiments to be queried, including accessing aggregate statistics for those predictions. In some implementations, a machine learning experiment can specify a computational model that is an ensemble of multiple models.

### CROSS REFERENCE TO RELATED APPLICATIONS

This application is a continuation application, claiming priority to, and the benefit of, PCT Application PCT/US22/28336, filed May 9, 2022, and entitled “PROCESSES, MACHINES, AND ARTICLES OF MANUFACTURE RELATED TO MACHINE LEARNING FOR PREDICTING BIOACTIVITY OF COMPOUNDS”, and designating the United States, which claims priority to, and the benefit of, U.S. Provisional Patent Application Ser. No. 63/186,274, filed May 10, 2021, and entitled “PROCESSES, MACHINES, AND ARTICLES OF MANUFACTURE RELATED TO MACHINE LEARNING FOR PREDICTING BIOACTIVITY OF COMPOUNDS”, all of which are hereby incorporated by reference.

### BACKGROUND

Machine learning generally involves using data about one set of items for which a property is known, such as classifications for the items, to train a computational model that in turn can make predictions about what that property should be for other items, for which that property is not known. While there is a wide range of possible applications of this general concept of machine learning, practical applications can be hard to implement for many reasons.

### SUMMARY

This Summary introduces a selection of concepts in simplified form that are described further below in the Detailed Description. This Summary neither identifies key or essential features, nor limits the scope, of the claimed subject matter.

Machine learning techniques can be used to build computer systems that can predict properties of items. To do so, the computer system has access to data representing a set of researched items for which a property is known. The property which a researched item has is one from among a plurality of types of properties. The computer system also has access to data representing potential candidate items. For each potential candidate item, respective information is not known for at least one property among the plurality of types of properties. The computer system applies machine learning techniques to train a computational model using the data representing the researched items and their known properties, for a plurality of types of properties. The computer system applies the trained computational model to the data representing the potential candidate items. In response, the trained computational model outputs one or more predictions about whether the potential candidate items are likely to have a property from among the plurality of types of properties that the computational model is trained to predict.

By way of illustration, an example implementation relates to predicting bioactivity of compounds. Machine learning techniques can be used to build computer systems that can predict bioactivity of compounds. To do so, the computer system has access to data representing a set of researched compounds for which bioactivity information is known. The bioactivity information for a researched compound characterizes bioactivity, of one or more types, in response to presence of the respective researched compound in or on a living thing. The bioactivity information can indicate the compound does, or does not, have a type of bioactivity and may include quantified information characterizing the bioactivity. The computer system also has access to data representing potential candidate compounds. Information which characterizes bioactivity, of one or more types, in response to presence of the respective potential candidate compound in or on a living thing, is not known. The computer system applies machine learning techniques to train a computational model using the data representing the researched compounds and their known bioactivity information for a plurality of types of bioactivity. The computer system applies the trained computational model to the data representing the potential candidate compounds. In response, the trained computational model outputs one or more predictions about whether the potential candidate compounds are likely to exhibit the bioactivity from among the plurality of types of bioactivity that the computational model is trained to predict.

The computer system can include an interface to define and generate multiple “machine learning experiments.” A machine learning experiment can be specified by a data structure called a “model set.” A model set includes data specifying a computational model, a selected subset of the researched items, and a selected subset of the plurality of potential candidate items. Researched items in the selected subset have information characterizing a known property of the researched item. The property which a researched item has can be one from among a plurality of types of properties.

Execution of a machine learning experiment provides a trained computational model. The trained computational model is applied to a selected subset of the plurality of potential candidate items to generate a respective result set for the model set. The result set comprises data representative of a set of predicted candidate items from among the plurality of potential candidate items. The trained computational model predicts, based on the selected subset of researched items, whether the predicted candidate items are likely to have one or more of the types of properties. The result set can include, for each predicted candidate item, a respective prediction value for the predicted candidate item for a type of property.

The computer system can include an interface through which an end user can specify machine learning experiments, by specifying data representing a computational model and specifying a subset of the plurality of researched items. One way the interface may permit a user to select researched items is through selecting one or more types of property. The computer system can use the selected one or more types of property to identify the researched items for which information for the selected one or more types of property is known. These identified researched items can form the selected subset of the researched items for the model set. Typically, a machine learning experiment would use data for a plurality of types of property, such that the researched items would include both positive and negative examples, i.e., items known to have, or not to have, the types of property. The interface of the computer system can further allow the end user to select from among the identified researched item to further refine the selected subset of the researched items.

Multiple result sets are generated by executing multiple different machine learning experiments. These multiple result sets can be used to define a database of predicted candidate items, with respective prediction values for respective predicted types of property. Different machine learning experiments each can result in different respective predictions that a predicted candidate item has a type of property. Specifically, one machine learning experiment can predict that an item has a type of property with a first prediction value, and another machine learning experiment can predict that this item has this type of property with a second prediction value. As a result, a predicted candidate item can have multiple prediction values for that type of property. Similarly, one machine learning experiment can predict that an item has a first type of property, and another machine learning experiment can predict that this item has a second type of property. Accordingly, a predicted candidate item can have prediction values for multiple types of property.

The computer system can have a query interface through which an end user, or other computer programs that can access the database, can query the result sets. Such queries can include sorting or filtering the predicted candidate items based on various characteristics, including aggregated statistics across several result sets or other information resulting from transformations of stored metadata about the predicted candidate items, or both.

For example, a query can identify a type of property. For example, the property may be a type of bioactivity, such as having an impact on a concentration of a protein. The computer system can access the database of result sets and identify any predicted candidate items that one or more machine learning experiments have predicted to have that property. The computer system can provide, as a result of accessing the database, data about the identified predicted candidate items, such as metadata related to the predicted candidate items from a database of items, or statistics about the predictions for the predicted candidate items, or other data, or any combination thereof.

For any predicted candidate item, several aggregate statistics can be computed about the predicted candidate item. The system can compute a function based on a number of machine learning experiments that predicted this predicted candidate item to have this type of property. The system can compute a function based on a number of types of properties that an item is predicted to have. The system can compute a function, such as a sum or average, based on the prediction values for the types of property that an item is predicted to have. Any one or more of these, and yet other statistics, can be computed from the database result sets.

For any type of property, several aggregate statistics can be computed about the type of property. The system can compute a function based on the items predicted to have this type of property, such as the number of items predicted to have this type of property. The system can compute a function based on the prediction values for the items predicted to have this type of property, such as an average prediction value across the items predicted to have this type of property. Any one or more of these, and yet other statistics, can be computed from the database result sets, and may be computed in combination about statistics computed about predicted candidate items.

Machine learning techniques are challenging to apply to this kind of data for several reasons, of which some are the following.

As described above, the training data set is selected from a set of researched items, such as researched compounds, for which quantifiable information about certain properties, such as bioactivity, is known. In many applications, the set of potential candidate items for which predictions are to be made, such as a set of potential candidate compounds, are collectively substantially different from the set of researched items, from a machine learning perspective.

There are several ways in which two sets of items can be different. For example, the distribution of values for a feature in the feature set used to describe the set of researched items may be different from the distribution of values for that feature for the potential candidate items. Such a problem is called “domain shift.” As another example, the supervisory information available for the researched items may be difficult to apply to the potential candidate items. As another example, there may be quality problems with the data about the researched items, or about the potential candidate items, or both, such as incompleteness, noise, or inconsistency. Each of these are described in more detail in the following paragraphs.

Such differences between researched items and the potential candidate items means that a computational model trained using the data about the researched items cannot be simply applied to the data about the potential candidate items. More specifically for compounds, several problems can arise when attempting to apply machine learning techniques using information about bioactivity of researched compounds to make predictions about bioactivity of potential candidate compounds. More specific examples are highlighted in the following.

As an example, in the context of compounds, when researched compounds are primarily synthetic molecules, or small molecules, and potential candidate compounds are naturally occurring, large molecules, the set of potential candidate compounds is collectively structurally different from the set of researched compounds. Specifically, the distribution of values for one or more features derived from the structures of molecules of the researched compounds may be substantially different from the distribution of values for the same features as derived from the structures of molecules of the potential candidate compounds.

As another example, in the context of compounds, data representing the researched compounds generally includes, for any given bioactivity, many examples of compounds that do not have the bioactivity (i.e., inactive compounds) and few examples of compounds that do have the bioactivity (i.e., active compounds). Such supervisory information, with few positive examples and many negative examples, is called “imbalanced.” Using imbalanced data for training a computation model tends to reduce the performance of the model, whether in training (e.g., leading to noise in monitoring convergence), or in use of a trained model (e.g., increasing the rate of false negative predictions). Using imbalanced data for training tends to introduce bias into a trained computational model. Specifically, the trained model may be overconfident in predicting negative results because it was not trained using enough relevant positive examples.

In some cases, data may be incomplete, noisy, or inconsistent. As an example, such problems often arise when data is received from different sources.

In the context of compounds, investigators from different laboratories may have reported, for the same compound, different measurements for a type of bioactivity. In some cases, the measurements may have arisen from substantially different laboratory experiments or assays, leading to “concept shift” between data points. In some cases, the measurements may have arisen from different implementations of substantially the same experiment or assay. But, where laboratory experiments are not entirely standardized or where, such as in the case of in vitro environments, laboratory experiments may not be entirely controllable, noise tends to be introduced into the measurements.

Another example of a problem arising when data is received from different sources is variation in format or reliability or quality of reported data. In the context of compounds, there are several examples. In some cases, reported bioactivity measurements may be truncated or censored or both.

When data is truncated, a measurement may be reported in a continuous format (e.g., a specific active concentration) when the measurement is on one side of a threshold, but in a binary or other discontinuous format (e.g., “inactive”) when the measurement is on the other side of that threshold. When truncated data is present in supervisory information used for training a computational model, there may be insufficient information to train a regression model on the truncated datapoints without additional inferential steps. For a classifier model, it becomes difficult to set thresholds for classes to fit the model.

When data is censored, a measurement may not be reported at all. In some cases, an experiment or assay may have been performed for a compound providing a measurement of bioactivity of that compound, but the measurement may not be reported. For example, the measurement may fall outside a range set by an investigator. In some cases, no experiment or assay is performed because an investigator believes the experiment or assay a-priori is unlikely to produce useful results. A large scale, untargeted, high-throughput screening program would likely have a low rate of compounds shown to have a type of bioactivity, and a large number of compounds indicated as inactive, and thus provides data that is more imbalanced. In contrast, a targeted study reported in literature would have censored data, resulting in a higher rate of compounds shown to have the type of bioactivity, compared to a large-scale screening program, and a smaller number of compounds indicated as inactive, and thus provides data that is more biased.

In general, publicly available bioactivity measurements may have a range of quality, and the quality of each source may be uncertain. Some assay protocols are more rigorously defined than others, and some assays have benefited from extensive iteration and improvement over time. Some laboratory environments are more well controlled and well equipped than others to produce repeatable and reliable measurements. Some data sources, such as the CHEMBL database, may include data that represents an attempt to assess and assign qualitative quality scores to bioactivity data.

Further, with such issues related to the data about researched items and potential candidate items, different computational models, training algorithms, training sets, and interventions to address these issues likely will produce different results, i.e., different models and differently trained models likely will make different predictions. Typically, an “optimal” model is sought by training and testing numerous models, but often finding an optimal model is not achievable.

To address the various machine learning problems that can arise, a platform, as described herein, allows multiple machine learning experiments to be defined, and then allows predictions from those multiple machine learning experiments to be queried to provide a set of nominations. The platform can generate aggregate statistics for the predictions made over multiple machine learning experiments, and those aggregate statistics can be used to filter, sort, select, and otherwise process the set of nominations.

This use of aggregate information about predictions made by different machine learning experiments eliminates the effort of trying to find an optimal model for making predictions. Instead, multiple different machine learning experiments can be defined, using differing computational models, training sets, training algorithms, and interventions to address issues due to the data. When predicting bioactivity of compounds, by using a variety of different statistics, sorting, and filtering, the nominations are more likely to identify predicted candidate compounds having a higher likelihood of actual bioactivity if appropriate laboratory experiments are performed to verify the predicted bioactivity. This enables prioritization of further experimentation on the predicted candidate compounds.

Further, to address the various machine learning problems that can arise, a variety of techniques can be used in this platform, whether alone or in combination, for use within the multiple machine learning experiments. These techniques can be implemented within the machine learning experiments, such as in the implementations of the computational models, in the implementations of the training algorithms for these computational models, or in the selection of training sets, or in how the outputs of different computational models are evaluated whether individually, or any combination of these. The implementations of the computational models can include how features are extracted from the input data. The implementations of the training algorithms can include how supervisory information is extracted from the training set.

In some implementations, a machine learning experiment can specify a computational model that is an ensemble of multiple models. Each model in a plurality of models has a respective output. The outputs of the multiple models are input to an ensemble function, which provides a final output of the computational model. Execution of the machine learning experiment for which the computational model is an ensemble of multiple models results in a set of trained models and an ensemble function. In some implementations, parameters of the ensemble function also may be trained.

In such implementations, the trained computational model, when applied to a selected set of potential candidate items, produces a result set in which each of the multiple models, and the ensemble function, provides information relevant to any prediction made for any potential candidate item.

In such implementations, the result set comprises data representative of a set of predicted candidate items from among the selected set of potential candidate items. The information stored for each of the predicted candidate items can include not only a prediction value for the predicted type of property, but other data provided by the multiple models and the ensemble function.

In some implementations, a machine learning experiment can specify a computational model that incorporates uncertainty modeling. Uncertainty modeling relates to discounting predicted activity of a primary model by predictions of a secondary model or through specialized post-processing of the predictions of the primary model. The secondary model can be any uncertainty model that can assess the reliability of the primary model.

An uncertainty model can be in itself a computational model that outputs its own prediction value. The input features for the uncertainty model can be derived in several ways, such as one or more of the following techniques. For example, the input features can be generated using various embedding techniques, such as autoencoders or other transforms, based on the data about the items processed by the primary model. The input features may include the output predictions of the primary model. The input features can include all or a subset of the input features of the primary model. Herein the prediction value output by the uncertainty model is called the “uncertainty value” to distinguish it from the prediction value output by the primary model of which reliability is being assessed.

In such implementations, both the uncertainty value for an item and a type of property as output by the uncertainty model and the prediction value for the item and the type of property as output of the computational model can be used to evaluate predicted candidate items. For example, the uncertainty value for an item and a type of property as output by the uncertainty model can be combined with the prediction value for the item and the type of property as output of the computational model. Uncertainty values, or other data computed based on uncertainty values, can be included in nominations of predicted candidate items, and can be used to sort and filter such nominations. In the context of compounds and other items for which properties are capable of scientific experimentation and validation, the uncertainty model and corresponding uncertainty value is intended to enable enhanced prioritization of predicted candidate items for lab validation.

In some implementations, a machine learning experiment can specify a computational model that incorporates sample weighting. Sample weighting addresses the problem of domain shift. Sample weighting involves upweighting samples close to the target domain during training. Class imbalance is addressed by equalizing the class weight of the training samples. Metrics used for sample weighting also can be reported for predicted items to help filter and sort predicted items.

In some implementations, model ensembles and uncertainty modeling are combined. In some implementations, model ensembles and sample weighting are combined. In some implementations, uncertainty modeling and sample weighting are combined.

The following Detailed Description references the accompanying drawings which form a part this application, and which show, by way of illustration, specific example implementations. Other implementations may be made without departing from the scope of the disclosure.

### BRIEF DESCRIPTION OF THE DRAWINGS

FIG. 1 is a data flow diagram of an example implementation of a computer system that uses machine learning techniques to predict bioactivity of compounds.

FIG. 2 is an example implementation of a data structure for data representing compounds, known bioactivity, and predicted bioactivity.

FIG. 3 is an example implementation of a data structure for data specifying machine learning experiments.

FIG. 4 is a data flow diagram of an example implementation.

FIG. 5 is an example implementation of a data structure for reporting predictions.

FIG. 6 is a block diagram of an example general purpose computer.

FIG. 7 is a data flow diagram of an example implementation of a computer system that uses an ensemble of models to predict bioactivity of compounds.

FIG. 8 is a flowchart describing an example operation of an ensemble of models.

FIG. 9 is a data flow diagram of an example implementation of a computer system that incorporates uncertainty modeling.

FIG. 10 is a flowchart describing an example operation of a computational model that incorporates uncertainty modeling.

FIG. 11 is a data flow diagram of an example implementation of a computer system that incorporates sample weighting.

FIG. 12 is a flowchart describing an example operation of a computational model that incorporates sample weighting.

In the drawings, in the data flow diagrams, a parallelogram indicates an object that is an input to a system that manipulates the object or an output of such a system, whereas a rectangle indicates the system that manipulates that object.

### DETAILED DESCRIPTION

Machine learning techniques can be used to build computer systems that can predict properties of items. To do so, the computer system has access to data representing a set of researched items for which a property is known. The property which a researched item has is one from among a plurality of types of properties. The computer system also has access to data representing potential candidate items. For each potential candidate item, respective information is not known for at least one property among the plurality of types of properties. The computer system applies machine learning techniques to train a computational model using the data representing the researched items and their known properties, for a plurality of types of properties. The computer system applies the trained computational model to the data representing the potential candidate items. In response, the trained computational model outputs one or more predictions about whether the potential candidate items are likely to have a property from among the plurality of types of properties that the computational model is trained to predict.

Items can include any of a variety of physical items, which may include machines, articles of manufacture, or compositions of matter, or any combination of these. Properties can include mechanical, optical, electrical, magnetic, electrooptical, electromagnetic, chemical, biological, or other properties (e.g., liquid, gas, solid, or other state) or any combination of these. Such physical items include compounds, and combinations of compounds, including various forms of such combinations (e.g., mixtures, solutions, alloys, conglomerates) or structure of such combinations (e.g., mechanical, electrical, or other interconnection). Further descriptions of example compounds and properties of compounds are provided below.

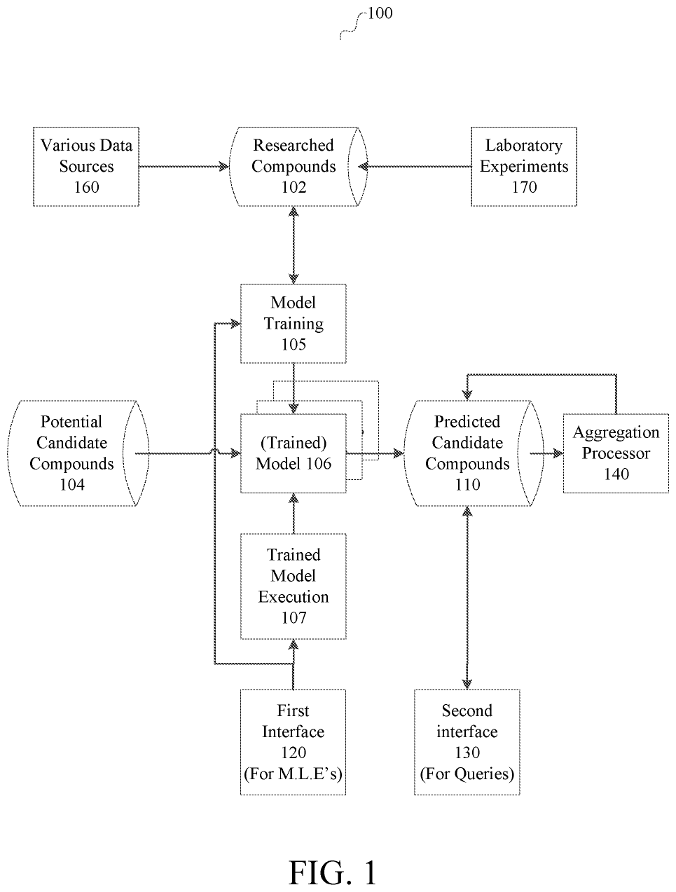

An example implementation relating to bioactivity of compounds is provided as an illustration. Referring now to the data flow diagram of FIG. 1, an example implementation of a computer system that uses machine learning techniques to predict bioactivity of compounds will now be described.

The computer system 100 has access to data 102 representing a set of researched compounds. A researched compound is a compound for which bioactivity information for a type of bioactivity is known. Bioactivity information is data characterizing a type of bioactivity in response to presence of a compound in or on a living thing. The computer system 100 also has access to data 104 representing potential candidate compounds. A potential candidate compound is a compound for which bioactivity information for a type of bioactivity is not known. Information about researched compounds can come from a various data sources 160, examples of which are described in more detail below, or from laboratory experiments 170, or both.

The computer system applies machine learning techniques, implemented by a model training system 105, to train a computational model 106 using data 102 representing the researched compounds and their known bioactivity information for a type of bioactivity. The computer system, using the trained model execution system 107, applies the trained computational model 106 to data 104 representing potential candidate compounds. In response, the trained computational model outputs data 110 representing one or more predictions about whether the potential candidate compounds are likely to exhibit the type of bioactivity.

The data 110 can include not only information from individual machine learning experiments, but also information resulting from processing data 110 received from multiple machine learning experiments to provide derived values or aggregate statistics for predicted candidate compounds, such as a number or percentage of types of bioactivity for which the compound is a predicted candidate compound. The data computed can include aggregated statistics across several result sets, or other information resulting from transformations of stored metadata about the predicted candidate compounds, or both. An aggregation processor 140, example implementations of which are described in more detail below, generates such derived values or aggregate statistics and stores them with the data 110. The aggregation processor can compute such data as a periodic background process, or as part of or after a machine learning experiment, or in response to a query, or as directed, or at any other time in any other manner. The computer system can include one or more interfaces for a user to interact with the system. A first interface 120, for which an example implementation is described below, allows a user to specify and execute machine learning experiments (“M.L.E.”), as described in more detail below, to create and execute trained models 106. A second interface 130, for which an example implementation is described below, allows a user to query the data 110 resulting from training and executing the trained model 106 through multiple machine learning experiments. In some implementations, the aggregation processor 140 can generate statistics in response to a query. In response to a query, the system presents nominations 150 indicative of predicted candidate compounds and associated statistics.

As used herein, a compound is any molecular structure. Compounds can be described by their source, such as a living thing, such as a plant or animal, naturally occurring or manufactured, industrial, pollutant, food, and so on. Compounds also can be described by their typical activity with respect to other compounds, such as binding, agonist, antagonist, increasing response, decreasing response, partial agonist, partial antagonist, inverse agonist/antagonist, transcription modulation, phosphorylation, sequestration, catalyst, and so on. Compounds can be described by their compositional type, such as small molecule, macromolecule, large molecule, or polymer. Molecules may be organic or inorganic. Example organic molecules include but are not limited to lipids, alkaloids, nucleic acids, polypeptides, and so on. Example polymers include but are not limited to proteins, peptides, nucleic acids (e.g., RNA, DNA, or fragments thereof), glycan, or any combinations of the above.

Non-limiting examples of properties of a compound include, but are not limited to physical properties, reactivity, bioactivity, or biological properties. Example physical properties include molecular weight, protonation state, salt state, melting point, crystal structure, boiling point, density, length, volume, pH, and so on. Examples of reactivity include side chains (e.g., OH, COOH, NH₂, etc.), a number of bonds, a number of rotatable bonds, and so on. Examples of biological properties include the source of the compound (e.g., plant, animal, fungus, etc.), metabolism, and so on.

As used herein, a living thing is any living thing, such as a plant or animal. Among animals, of most interest is mammals, especially mammals having similar biological systems as human beings. The living thing for which bioactivity of a compound is researched or predicted can be limited to humans, mammals, or any type, class, kind, genus, or species, of plant or animal. A kind of living thing for which bioactivity of a compound is predicted may be different from a kind of living thing for which the bioactivity of the compound is known. For example, bioactivity of a compound in a mouse may be useful in predicting bioactivity of that compound and yet other compounds in a human.

Bioactivity of a compound means any quantifiable biological response of a living thing when the compound is present in or on the living thing. The biological response can be quantified through in vitro or in vivo experiments or measurements. In vitro experiments can be limited to include cells of interest from a living thing. Any one or more of the following can further characterize the biological response. The biological response can be positive (i.e., healthy), negative (i.e., unhealthy), or neutral, or a combination of responses, such as a positive response such as reduction of a symptom and a simultaneous negative response such as a side effect. The biological response can include a first compound decreasing bioactivity related to a second compound, such as a drug. The biological response can include a first compound increasing bioactivity related to a second compound, such as a drug. The biological response may be a direct response to the compound or an indirect response to the compound. The biological response may be a conditional response to the compound, involving presence of one or more other compounds for the biological response to occur. The bioactivity may occur in an organ, a bodily fluid, or other part of a body.

The biological response can be related to a health condition of, or health treatment for, the living thing. The biological response can be related to a concentration of a protein present in the living thing. The biological response can be related to toxicity of the compound to the living thing. The biological response can be related to absorption, distribution, metabolism, or excretion related to the compound. The biological response can be related to factors that cause, reduce, or otherwise affect neoplasms or tumors, whether benign or malignant, such as cancers.

The biological response can be related to epigenetics. The biological response can be related to gene activity and expression. The biological response can be related to alterations of a DNA sequence such as by methylation, acetylation or deacetylation, phosphorylation or dephosphorylation. The biological response can be related to a signal pathway. The biological response can be related to change in a transcription factor. The biological response can be related to cytotoxicity, i.e., cell death (in contrast to toxicity to the living thing or tumors).

To be useful in a machine learning context, the property of an item is quantifiable. In the context of compounds and their bioactivity, the biological response is quantifiable. An example of a quantifiable biological response is a measured concentration of an item, such as a protein, in a sample in response to presence of a measured amount of the researched compound. Information that quantifies a biological response can be an amount in a continuous range, in a piece-wise continuous range, or in a discrete range. The information that quantifies a biological response generally results from an assay which measures a characteristic of a reaction of a compound with a sample. This information can represent, for example, a concentration of a protein, a concentration of another item related to an amount of a protein, a concentration of RNA expression data, a readout from a sensor, such as luminescence, fluorescence, or radiation, or any other characteristic of the reaction that can be measured.

A dense data set including many measurements of the biological response in response to different measured amounts of a compound is preferable. Examples of existing dense data sets include, but are not limited to: databases available through the Toxicology in the 21st Century (Tox21) Consortium (described in at least Attene-Ramos, Matias S., et al., “The Tox21 robotic platform for the assessment of environmental chemicals—from vision to reality.” Drug discovery today, 18.15-16 (2013): 716-723, at available through, herein called “the Tox21 database”), the ChEMBL database available from the European Bioinformatics Institute (described in at least Gaulton, Anna, et al, “The ChEMBL database in 2017,” Nucleic acids research 45.D1 (2017): D945-D954, and available through, herein called “the ChEMBL database”), or others, or any combination of these.

A compound may be present in or on a living thing in a variety of ways. For animals, a compound may be, for example, ingested by mouth, by inhaling, by injection, by being absorbed through skin, hair, mucous membrane, or other surface. The compound may become present because of some biochemical process applied to yet another compound. Examples of such biochemical processes include, but are not limited to one or more of metabolization, hydrolyzation, digestion, or any other biochemical process in the living thing. The compound may be present in a time-varying concentration due to biochemical processes. The compound can become present intentionally or knowingly, such as with a food or medicine, or can become present unintentionally, accidentally, negligently, or unknowingly, such as with contaminants.

Compounds can include a set of compounds that are naturally occurring in or on foods, such as compounds in or on vegetables, fruits, grains, other plants, land animals, whether wild, domesticated, hunted, or farmed, and seafoods, whether farmed or fished, and other compounds which may arise in food production. Some compounds may be in the category of compounds which are generally regarded as safe (GRAS) for human and livestock food production. Compounds can include non-foods that are intentionally introduced into a living thing, such as drugs, medicines, and vaccines. Compounds can include any compound occurring in the air, water, or ground, with which the living thing may come in contact. Compounds can include residues, contaminants, pollution, toxins, insecticides, fungicides, food additives, and other byproducts of food production, harvesting, manufacturing, preparation, packaging, transportation, distribution, storage, sale, or other activity. Compounds may be created by biochemical processes associated with the living thing being studied or other living things such as secretions by a microbiome. Compounds also may arise from chemical reaction processes independent of biology, e.g., as may be associated with degradation of a drug in the body.

Data representing a variety of compounds can be accessed from different sources and stored in the system. Example sources include: the FooDB database (described at least in Naveja, J Jesús et al., “Analysis of a large food chemical database: chemical space, diversity, and complexity,” F1000Research vol. 7 Chem Inf Sci-993. 3 Jul. 2018, doi:10.12688/f1000research.15440.2, available at https://foodb.ca/, and herein called “the FooDB database”), or others such as described in Barabasi, A., Menichetti, G. & Loscalzo, J. The unmapped chemical complexity of our diet. Nat Food 1, 33-37 (2020). https://doi.org/10.1038/s43016-019-0005-1, or any combination of these.

A compound can be a potential candidate compound with respect to one type of bioactivity, yet a researched compound with respect to another different type of bioactivity, and yet a predicted candidate compound with respect to yet another different type of bioactivity. A compound can be at one time a potential candidate compound, and then that compound can become a predicted candidate compound for a selected bioactivity. Laboratory experiments can be performed on the predicted candidate compound. The computer system 100 can have an input interface (not shown) through which data can be received that includes information characterizing verified bioactivity based on laboratory experiments 170 performed with a predicted candidate compound. Through this interface, such data can be stored in the database 102 of researched compounds, thus making the predicted candidate compound a researched compound with respect to that bioactivity.

Data representing an item typically includes features sufficient to distinguish the item from other items in the same class by virtue of its inherent structure or composition, source, and/or impacts on one or more aspects of the modeled system. For example, data representing a compound includes at least data from which features can be derived for the compound. For machine learning, such features are used for training a computational model using, or for applying a trained computational model to, the data representing the item. The features may be a part of the data representing the item or may be derived from other data representing the item.

For compounds, such data typically includes data defining the molecular structure of the compound. Data defining molecular structure of a compound can include any one or more of data representing: a molecular formula for the compound, a name for the compound, any isomers of the compound, a two-dimensional chemical structure of the compound, a SMILE string, three dimensional conformations of the molecule of the compound, any chemical property descriptors such as an RDKit descriptor, molecular properties, such as crystal structure, molecular weight, solubility, or any features resulting from transformation of such information which can be input to a machine learning module. Data defining a compound can include a mapping onto a protein-protein interaction graph based on known compound-protein interactions, which is an ‘impact’-based featurization. As another example, for functional RNA data defining a compound can include an inherent composition based on primary sequence and secondary structure such as the presence of certain key motifs and kmers, or a transcriptomic differential expression profile when the functional RNA is perturbing a basal cell line, or other representation, or any combination of these.

In many applications where predictions are to be made about potential candidate compounds based in information about researched compounds, the plurality of potential candidate compounds typically are “collectively structurally different” from the plurality of researched compounds. For example, researched compounds for which information characterizing bioactivity is available typically are synthetic compounds, such as compounds found in drugs, pharmaceuticals, medicine, processed food products, or other sources, and are typically, but not necessarily, small molecules or peptides. The potential candidate compounds typically are naturally occurring compounds, such as compounds found in foods, plants, and animals, which are typically, but not necessarily, larger molecules.

Yet additional examples of compounds include but are not limited to various small molecules and macro molecules. A non-limiting example ontology of small molecules is described in the Dictionary of Natural Products, available from CRC Press. Examples of macromolecules includes, but is not limited to polypeptides, nucleic acid sequences, and lipids. Examples of nucleic acid sequences includes but is not limited to RNA, coding RNA, non-coding RNA, DNA, including fragments of such sequences, such as cell-free DNA.

Some examples of qualitative and quantitative differences between sets of compounds include, but are not limited to: differences in the nature, presence, or type of features that can be derived from their data; differences in the distribution of molecular descriptors such as molecular weight; differences in the distribution of the presence of certain chemical scaffolds such as aromatic rings; differences in variance in chemical structures among compounds. As an example, drug compound libraries often have numerous compounds that are small variations on a common backbone, whereas food compound libraries often have many compounds that do not have common structures. Compounds that are naturally occurring in foods tend to have significant scaffold diversity, structural complexity, molecular mass, and molecular rigidity. Such compounds also tend to have a larger number of sp3 carbon atoms and oxygen atoms but fewer nitrogen and halogen atoms, higher numbers of H-bond acceptors and donors, lower calculated octanol—water partition coefficients indicating higher hydrophilicity, than synthetic small molecules.

A molecular descriptor either is a result of a logical or mathematical procedure which transforms chemical information encoded within a symbolic representation of a molecule into a useful number, or is a result of a standardized experiment that measures a quantity related to the molecule, or a combination of both. Molecular descriptors can be computed in many ways. For example, molecular descriptors can be a property of a molecule that can be calculated or approximated from its molecular formula or SMILES string. such as its weight, solubility, charge, or aspects of its shape. For some molecules, parameters such as a sequence length or entropy can be useful. See, e.g., “MathFeature: feature extraction package for DNA, RNA and protein sequences based on mathematical descriptors,” by Robson P. Bonidia, Douglas S. Domingues, Danilo S. Sanches and André C.P.L.F. de Carvalho, in Briefings in Bioinformatics, 23(1), 2022, 1-10. A software package called RDKit is publicly available and includes implementations of computations of certain molecular descriptors. Molecular descriptors can be used as features and may be referred to herein as molecular features.

An example use case that will be used primarily in this description is where the researched compounds include mostly small molecules of drugs and pharmaceuticals, and the potential candidate compounds are molecules found in foods and food products, whether naturally occurring or not, especially any compounds that are generally recognized as safe (GRAS). Data about these two sets of compounds could be used, for example, to: identify food compounds that have similar bioactivity as drugs; identify food compounds that enhance bioactivity of drugs; identify food compounds that interfere with bioactivity of drugs; or identify combinations of such food compounds. These two sets of compounds are but one example of sets that are “collectively structurally different” from each other.

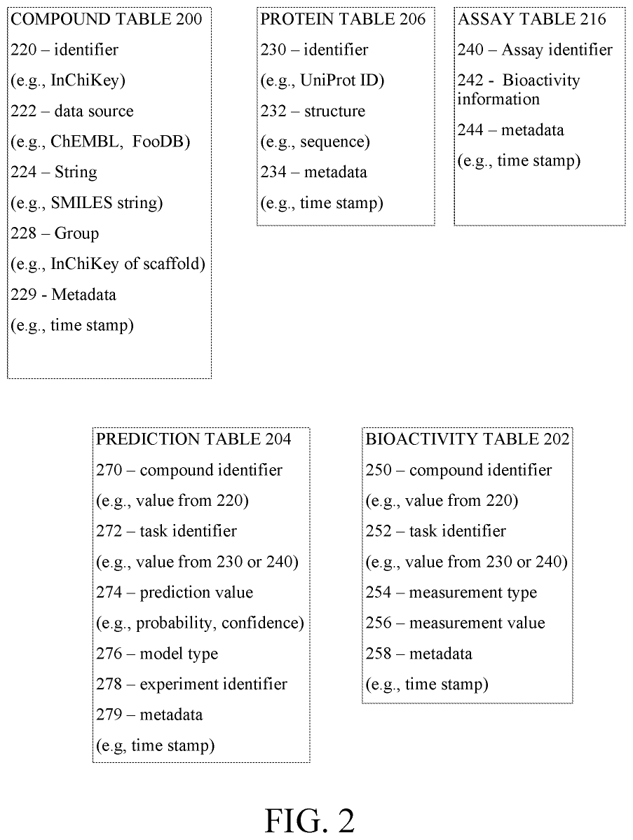

Illustrative data structures for an example implementation of such a computer system for the purposes of researching compounds are shown in FIG. 2. Such data structures can be implemented, for example, using one or more tables in a relational database, or using one or more data objects in an object-oriented database, or using one or more documents in a NoSQL database, or by using data structures allocated in memory for an executing computer program, or by using any data structures implemented through other programming techniques. The use of database tables in the following examples is merely illustrative.

As shown in FIG. 2, a compound table 200 can be used to represent all compounds, whether researched compounds, potential candidate compounds, or predicted candidate compounds. Another table, herein called a bioactivity table 202, includes data representing information characterizing bioactivities of compounds. Thus, if a compound has known bioactivity, making it a researched compound with respect to that type of bioactivity, then the compound has an entry in the bioactivity table 202 which includes information characterizing that bioactivity. Another table, herein called a prediction table 204, includes data representing predicted bioactivity for compounds. Thus, if a compound has been predicted to have bioactivity, making it a predicted candidate compound with respect to that type of bioactivity, then the compound has an entry in this table that includes information, herein called prediction data, describing that prediction.

In the example shown in FIG. 2, one kind of bioactivity that can be represented in the bioactivity table 202 and the prediction table 204 is a compound-protein interaction. The bioactivity of interest is the impact a compound may have on the production of a protein in a living thing. In FIG. 2, a protein table 206 includes data representing proteins, with each protein having a protein identifier. A similar assay table 216 can be defined for other kinds of bioactivity, with each type of bioactivity having an assay identifier, identifying an assay used to measure the bioactivity.

In the example of a compound-protein interaction, the bioactivity table 202 includes entries, with each entry associating a compound with a respective protein, and the quantifiable data describing that compound-protein interaction. For other types of bioactivity, the bioactivity table 202 includes entries, with each entry associating a compound with a respective bioactivity and data describing the associated quantifiable biological response.

In the example of a compound-protein interaction, the prediction table 204 includes entries, with each entry associating a compound with a respective protein for which it is predicted to have bioactivity, and data about that prediction. For other types of bioactivity, the prediction table 204 includes entries, with each entry associating a compound with a respective bioactivity for which a prediction has been made, and data describing that prediction.

Additional details of an example implementation of the tables in FIG. 2 will now be described. It should be understood that this example is merely illustrative, as a variety of information can be stored in the database in diverse ways.

In this example, compound table 200 includes data representing each compound. For each compound, information such as an identifier 220, can be stored. This identifier can be used as a primary join key with other tables. As an example, a suitable identifier is a form of an International Chemical Identifier (InChi) of the compound, such as the InChi identifier or the InChiKey identifier of the compound. For nucleotides, a suitable equivalent of the SMILES or InChi identifiers is the sequence. An equivalent of the InChiKey, in terms of being a unique identifier, is the NCBI GI number or Uniprot ID. The InChi or InChiKey represents various information about the compound, including chemical formula, connectivity, stereochemistry, and charge. The InChiKey can be used as a primary key in the database for uniquely identifying compounds. In use cases that do not distinguish among different stereoisomers and charge, another identifier can be used. For example, a first segment of the InChiKey contains only chemical formula and connectivity information, but not charge or stereochemistry information. One or more of such identifiers can be stored, allowing processing of the table in diverse ways.

The data representing a compound can include an indicator 222 of a source from which information about the compound has been obtained. In the example of FIG. 2, a flag can indicate whether information about the compound was obtained from the ChEMBL database, the Tox21 database, or the FooDB database, or other database, or any combination of these, or none of these.

The data representing a compound can include a string 224 of characters describing the chemical structure of the compound. For example, the string can be in a format compliant with the “simplified molecular-input line-entry system” (SMILES) specification, commonly called a SMILES string. SMILES strings are advantageous because most molecule editing software allows import and export of SMILES strings to create two-dimensional drawings or three-dimensional models of molecules. One or more of such strings can be stored. For example, if data about a compound is imported from a source, and that source provides a SMILES string, then the original string can be stored. This original string can be converted to a canonical form, and this canonical form can be stored. The original string, or the canonical string, or any other suitable string, or any combination of these, can be stored. The string can directly or indirectly provide information about the compound. For example, the string may include data defining chemical structure of the compound. For example, the string may be a reference to a data file defining chemical structure or other information about the compound. Other data or file formats that define a compound can be used, which include but are not limited to: an MDL Molfile file or other chemical table (CT) files, or a chemical markup language (CIVIL) file.

The data representing a compound can include group information 228. A plurality of compounds can be placed into a group. A plurality of different groups can be defined. A compound can be placed into one or more groups. For example, the InChiKey of a scaffold for a compound can be used to group compounds. Grouping of compounds enables other advantageous operations to be performed in the context of training and using computational models. For example, when specifying train/validate/test splits, placing members of the same scaffold family into the same split tends to reduce overestimating generalization, because predictions for members of the same scaffold family are expected to be similar.

Other metadata 229 about the data representing the compound can be stored. For example, a time stamp can be stored indicating the last time the data representing the compound was modified. A variety of other metadata can be stored. For example, metadata about provenance of data stored in the system can be included, in addition to its source 222.

In the example shown in FIG. 2, a protein table 206 includes data representing each protein (the production or suppression of which may be a bioactivity in response to presence of a compound in or on a living thing). For each protein, the data representing the protein includes an identifier 230. This identifier can be used as a primary join key with other tables. An example identifier is an identifier for the protein as used in the UniProt database, also called the “UniProt ID.” Any other suitable identifier that uniquely identifies the protein can be used.

For each protein, the data representing the protein can include data 232 defining the chemical structure of the protein, such as a sequence for the protein. Any other information about the protein can be stored in the database, such as information about the protein from the UniProt Knowledgebase (UniProtKB) database or the SIFTER database, or Pubchem database, or other database, or any combination of these.

Other metadata 234 about the data representing the protein can be stored. For example, a time stamp can be stored indicating the last time the data representing the protein was modified.

In the example shown in FIG. 2, an assay table 216 includes data representing any other type of bioactivity in response to presence of a compound in or on a living thing. For each type of bioactivity, the data representing the bioactivity includes an assay identifier 240. The identifier can be any way of uniquely identifying an assay used to measure this bioactivity. This identifier can be used as a primary join key with other tables. For each type of bioactivity, the data representing the bioactivity can include any other useful information 242 about the bioactivity. Other metadata 244 about the data representing the protein can be stored. For example, a time stamp can be stored indicating the last time the data representing the protein was modified.

In the example shown in FIG. 2, bioactivity table 202 includes data representing known bioactivity of a compound, which is implemented by a table pairing identifiers of compounds with identifiers of types of bioactivities, such as a protein identifier 230 or assay identifier 240. A compound identifier field 250 stores the identifier 220 of the compound; the task identifier field 252 stores, for example, either a protein identifier 230 or an assay identifier 240. Bioactivity table 202 further associates such pairings with information characterizing the bioactivity. For example, data indicating a type 254 of measurement or assay or experiment used, and any value 256 resulting from that measurement or assay or experiment can be stored. Other metadata 258 about the known bioactivity can be stored. For example, a time stamp can be stored indicating the last time this data was modified.

In the example shown in FIG. 2, prediction table 204 includes data representing predicted bioactivity of a compound, which is implemented by a table pairing identifiers of compounds with identifiers of types of bioactivities, such as a protein identifier 230 or assay identifier 240. Data in the prediction table 244 are populated as a result of a “machine learning experiment”, which involves training a selected model using data about selected researched compounds and a selected type of bioactivity, and then applying the trained model to data about selected potential candidate compounds. The system allows for multiple such machine learning experiments to be specified and executed to generate the data about predicted bioactivity in this table. The specifications for such machine learning experiments are referred to as “model sets” and are described in more detail below.

In prediction table 204, a compound identifier field 270 stores the identifier 220 of the compound; the task identifier field 272 stores, for example, a protein identifier 230 or an assay identifier 240. Prediction table 204 further associates such pairings with information characterizing the prediction about the bioactivity. For example, this information can include a prediction value 274 of the bioactivity for the compound, and a class 276 indicating a type of machine learning model that generated the prediction, to help interpret the prediction value. Different types of machine learning models generate different kinds of prediction values 274, such as a probability, a confidence, a classification, or other output or combination of outputs. An identifier 278 of the machine learning experiment (as described below) that resulted in this prediction also can be stored. Other metadata 279 about the prediction can be stored. For example, a time stamp can be stored indicating the last time this data was modified.

The specification of “machine learning experiments” which access and use the data in the compound table 200, bioactivity table 202, protein table 206, and assay table 216, to generate the data in prediction table 204, will now be described in more detail by way of an example, illustrative implementation.

A machine learning experiment takes a computational model and a training set of data, e.g., data about researched compounds, and trains the computational model using a training algorithm, features derived from the training set, and supervisory information available for or derived from the training set. The trained computational model is then applied to a target data set, e.g., a set of potential candidate compounds, to make predictions about the target data set.

A computational model used in a machine learning application typically computes a function of a set of input features, which may be a linear or non-linear function, to produce an output. The function typically is defined by mathematical operations applied to a combination of a set of parameters and the set of input features. Machine learning involves adjusting the set of parameters to minimize errors between the function as applied to a set of input features for a set of training samples and known outputs (supervisory information) for that set of training samples. The output of the computational model typically is a form of classification or prediction.

Such computational models are known by a variety of names, including, but not limited to, classifiers, decision trees, random forests, classification and regression trees, clustering algorithms, predictive models, neural networks, genetic algorithms, deep learning algorithms, convolutional neural networks, artificial intelligence systems, machine learning algorithms, Bayesian models, expert rules, support vector machines, conditional random fields, logistic regression, maximum entropy, among others.

Some specific examples of models designed for use with compounds include, but are not limited to, the following. Some models make predictions based on expert-designed molecular descriptor features. These descriptors are intended to integrate prior knowledge of the feature space reflected by domain expertise, such as a deterministically computable molecular properties like charge and weight or the presence of certain subgroups known to be associated with bioactive effects.

Some models employ graph convolutional architectures. An example of such a model is described in Wu, Zhenqin, et al. “MoleculeNet: a benchmark for molecular machine learning,” Chemical science 9.2 (2018): 513-530, which is also available as the DEEPCHEM software package through https://deepchem.io (hereinafter “DeepChem”).

Some models incorporate edge features in molecular graphs (i.e., bonding information). An example of such a model is described in Yang, Kevin, et al. “Analyzing learned molecular representations for property prediction.” Journal of chemical information and modeling 59.8 (2019): 3370-3388 (hereinafter “ChemProp”).

Some models incorporate sequence data about proteins. Examples of such protein sequence models are described in Karimi, Mostafa, et al. “DeepAffinity: interpretable deep learning of compound—protein affinity through unified recurrent and convolutional neural networks.” Bioinformatics 35.18 (2019): 3329-3338 (hereinafter “DeepAffinity”), and in Li, Shuya, et al. “MONN: A Multi-objective Neural Network for Predicting Compound-Protein Interactions and Affinities.” Cell Systems 10.4 (2020): 308-322 (hereinafter “MONN”).

Some models are supervised with information about the binding sites of individual atoms on proteins. An example of a protein binding model also is described in MONN.

Examples of other models include, but are not limited to, those described in Zhavoronkov, A., Ivanenkov, Y. A., Aliper, A. et al., “Deep learning enables rapid identification of potent DDR1 kinase inhibitors,” in Nat. Biotechnol. 37, 1038-1040 (2019), or in Westerman, Kenneth E., et al. “PhyteByte: Identification of foods containing compounds with specific pharmacological properties,” in BMC Bioinformatics 21.1 (2020): 1-8.

In some use cases described herein, the output of a computational model is a prediction value indicative of whether or to what extent a compound has, if any, a selected type of bioactivity. This prediction can be in the form of, for example, a probability between zero and one, or a binary output, or a score (which may be compared to one or more thresholds), or other format. The output can be accompanied by additional information indicating, for example, a level of confidence in the prediction. The output typically depends on the kind of computational model used.

As described herein, each implementation of a particular kind of computational model can be assigned an identifier. The identifier can be mapped to the computer program instructions used to execute the computational model, such as a path in a file system to an executable program. Thus, a machine learning experiment can be defined in part by specifying a particular implementation of a computational model to be used.

A training set generally comprises a set of samples for which respective information about each sample is known, i.e., a set of researched compounds. Data called “features” are derived from information available about the samples the training set. These features are used as inputs to a computational model. The known information for the samples, typically called “labels,” i.e., the information characterizing the known bioactivity of the researched compounds, provides the supervisory information for training. The supervisory information typically corresponds to the desired outputs of a computational model. A computational model has parameters that are adjusted by the training algorithm so that the outputs of the computational model, in response to the features for the samples in the training set, correspond to the supervisory information for those samples. Most training algorithms divide the training set into training data and validation data. Given a trained computational model, the trained computational model can be applied to features derived from the data for potential candidate compounds. The trained computational model provides an output indicative of a prediction about the potential candidate compound.

In the example shown in FIG. 2, data for a training set can be specified by a query (or an identifier for such a query) on the compound table 200 joined with entries from the bioactivity table 202 that contain one or more selected values (e.g., protein identifiers or assay identifiers) as the task identifier. Similarly, data for the potential candidate compounds can be specified by a query (or an identifier for such a query) on the compound table 200 where those compounds are not in the training set and satisfy any other criteria desired.

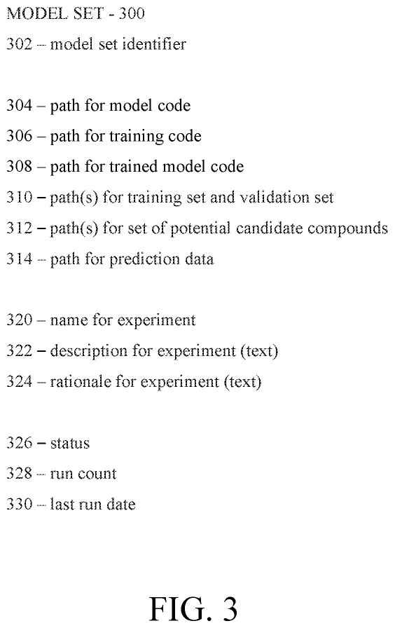

Therefore, as an example implementation shown in FIG. 3, a machine learning experiment can be specified by a data structure called herein a model data set 300. The model data set has an identifier 302 that uniquely identifies the model data set from among other model data sets within the system. The model data set 300 can include several data paths identifying locations for executable code and data. For example, there can be data paths that identify where (304) executable code for a computational model is stored, where (306) executable code for a training algorithm is stored, where (308) executable code for the resulting trained computational model will be stored, where (310) data defining training and validation sets (data for researched compounds) are stored, where (312) data defining a test set (the potential candidate compounds) are stored, and where (314) data representing predictions will be stored. Such data may be initially separate from the database shown in FIG. 2, and then may be uploaded into the database in FIG. 2.

The data representing the machine learning experiment can include other data fields, such as human-readable data about the machine learning experiment, and status information. Examples of human-readable data include, but are not limited to one or more of: a name 320, such as an alphanumeric string, a description 322 as a free text, human-readable description of the machine learning experiment, a rationale 324 as a free text, human-readable rationale for why this experiment was done or what was hoped it would prove or show. Examples of status information include, but are not limited to one or more of: status 326 of the experiment, a number of runs 328 of the experiment, a last run date 330 for the experiment, etc.

Referring to 310 and 312, the data identifying the researched compounds and the potential candidate compounds can be in many forms. In some implementations, the data identifying these sets of compounds can be a reference to where data for the sets are stored, such as a path and filename for a data file. In some implementations, the data identifying these sets of compounds can be a reference to where computer program code for accessing the sets of data is stored, such as a data file specifying a query.

In some implementations, the researched compounds can be specified by a query on the database of compounds. Such a query identifies at least one or more types of bioactivity, allowing compounds with that type, or those types, of bioactivity, and the quantified data characterizing that bioactivity, to be identified. The potential candidate compounds can be specified by any query that identifies compounds which do not have the specified one or more types of bioactivity.

In some implementations, the data for the researched compounds is extracted from the database into separate data storage for use in training. In some implementations this data is transformed into features which are stored in separate data storage for use in training. In some implementations, the data for the potential candidate compounds is extracted from the database into separate data storage for use in making predictions. In some implementations this data is transformed into features which are stored in separate data storage for use in making predictions.

Some machine learning experiments can be defined for a single type of bioactivity, or target. Some machine learning experiments can be defined for multiple types of bioactivity, or targets. When there are multiple targets, there can be an independent computational model used for each target, or there can be one computational model that makes predictions for all targets. When using a single model for multiple targets, information can be pooled. Learning on multiple targets simultaneously can increase the performance of the model on any one target. In models that integrate protein sequence information as described herein, by training on multiple targets, the models can generalize to novel protein sequences by learning feature response surfaces from other protein targets. Some other models that are multi-task models still pool information but do so in learning the compound representation layers. When using an independent model for each target, the system remains linearly scalable, in that there is a fixed runtime per target for a given sample size, and the introduction of bias is reduced.

Given a specification of a machine learning experiment, such as through a model set as shown in FIG. 3, the machine learning experiment can be executed. For example, the model training system 105 (FIG. 1) can train a computational model using the data representing the selected subset of the plurality of researched compounds from the database. The model training system implements the training algorithm specified by the model set. Generally, a training algorithm applies, as inputs to the computational model, features derived from the data representing the selected subset of the plurality of researched compounds. Outputs from the computational model are obtained and compared to the supervisory information corresponding to those inputs. Parameters of the computational model are modified so as to reduce the errors between the outputs obtained and the supervisory information. The training algorithm involves iterating these steps of applying, comparing, and modifying until such errors are sufficiently reduced.

After the computation model is trained, the trained model execution system 107 applies the trained computational model 106 to the data representing at least a subset of the plurality of potential candidate compounds. The trained computational model thus generates and stores a result set for the model set. The result set includes a set of predicted candidate compounds (110 in FIG. 1) identified from among the plurality of potential candidate compounds as likely to have the selected type of bioactivity. Such information can be stored, for example, in a data structure such as shown as a prediction table 204 in FIG. 2. In some implementations, and depending on the type of trained computational model and its output, one or more tests can be applied to the output produced by the trained computational model in response to the data representing a potential candidate compound to determine whether or to what extent the potential candidate compound is predicted to have the selected type of bioactivity. For example, one or more thresholds can be applied to the output so that only selected ones of the potential candidate compounds have information stored in the prediction table 202 indicating that the compound is now a predicted candidate compound for that bioactivity.

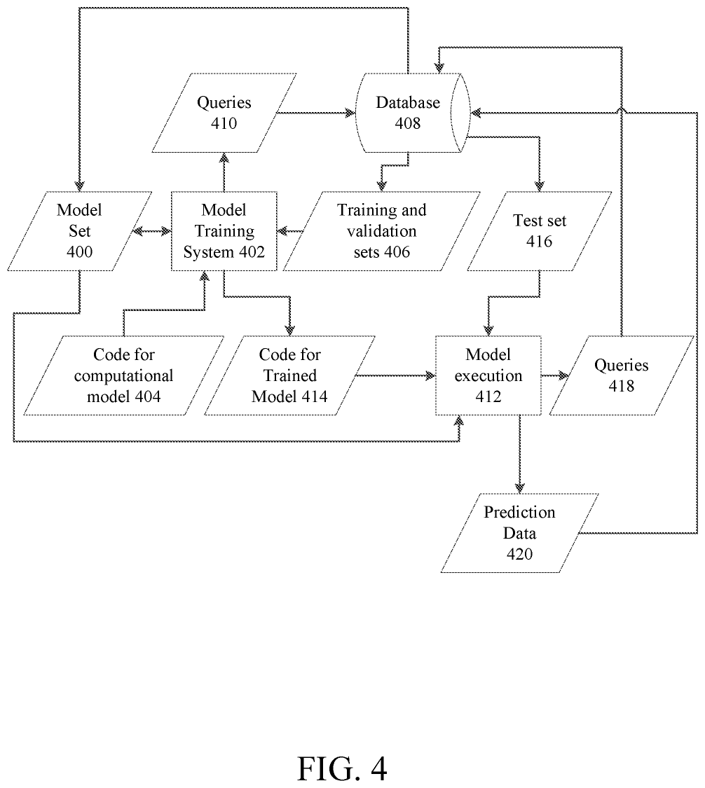

A data flow diagram illustrating more details for an example implementation of a system including a model training system and a trained model execution system will be described now in connection with FIG. 4.

In FIG. 4, a model set 400 (such as the data structure of FIG. 3) is accessed by the model training system 402. The model training system uses the data in the model set 400 to access code 404 to be used for implementing and training a computational model and to access training and validation sets 406 from the database 408. Accessing the data sets 406 may involve generating and running queries 410 on the database 408. The model training system 402 then trains the computational model, to generate code 414 defining the trained model. This code is stored in a file accessible at a path specified in the model set 400.

A model execution system 412, using the data from the model set 400, accesses the code 414 for the trained model and data for a test set 416. The model execution system 412 may generate and run queries 418 on the database 408 to access the data for the test set 416. The code 414 for the trained model is executed on the test set 416 to generate prediction data 420, which may be initially stored in a data file at a path specified in the model set 400, which in turn can be stored in the database 408.

As described above, the training data set is selected from a set of researched items, such as researched compounds, for which quantifiable information about certain properties, such as bioactivity, is known. In many applications, the set of potential candidate items for which predictions are to be made, such as a set of potential candidate compounds, are collectively substantially different from the set of researched items, from a machine learning perspective.

Also, as described above, there are several ways in which two sets of items can be different. For example, the distribution of values for a feature in the feature set used to describe the set of researched items may be different from the distribution of values for that feature for the potential candidate items. Such a problem is called domain shift. As another example, the supervisory information available for the researched items may be difficult to apply to the potential candidate items. As another example, there may be quality problems with the data about the researched items, or about the potential candidate items, or both, such as incompleteness, noise, or inconsistency. Further examples of quantitative and qualitative differences between sets of compounds are provided above.

Such differences between researched items and the potential candidate items means that a computational model trained using the data about the researched items cannot be simply applied to the data about the potential candidate items. More specifically for compounds, several problems can arise when attempting to apply machine learning techniques using information about bioactivity of researched compounds to make predictions about bioactivity of potential candidate compounds. More specific examples are highlighted in the following.

As an example, in the context of compounds, when researched compounds are primarily synthetic molecules, or small molecules, and potential candidate compounds are naturally occurring, large molecules, the set of potential candidate compounds is collectively structurally different from the set of researched compounds. Specifically, the distribution of values for one or more features derived from the structures of molecules of the researched compounds may be substantially different from the distribution of values for the same features as derived from the structures of molecules of the potential candidate compounds.

As another example, in the context of compounds, data representing the researched compounds generally includes, for any given bioactivity, many examples of compounds that do not have the bioactivity (i.e., inactive compounds) and few examples of compounds that do have the bioactivity (i.e., active compounds). Such supervisory information, with few positive examples and many negative examples, is called “imbalanced.” Using imbalanced data for training a computation model tends to reduce the performance of the model, whether in training (e.g., leading to noise in monitoring convergence), or in use of a trained model (e.g., increasing the rate of false negative predictions). Using imbalanced data for training tends to introduce bias into a trained computational model. Specifically, the trained model may be overconfident in predicting negative results because it was not trained using enough relevant positive examples.

In some cases, data may be incomplete, noisy, or inconsistent. As an example, such problems often arise when data is received from different sources.

In the context of compounds, investigators from different laboratories may have reported, for the same compound, different measurements for a type of bioactivity. In some cases, the measurements may have arisen from substantially different laboratory experiments or assays, leading to “concept shift” between data points. In some cases, the measurements may have arisen from different implementations of substantially the same experiment or assay. But, where laboratory experiments are not entirely standardized or where, such as in the case of in vitro laboratory environments, experiments may not be entirely controllable, noise tends to be introduced into the measurements.

Another example of a problem arising when data is received from different sources is variation in format or reliability or quality of reported data. In the context of compounds, there are several examples. In some cases, reported bioactivity measurements may be truncated or censored or both.

When data is truncated, a measurement may be reported in a continuous format (e.g., a specific active concentration) when the measurement is on one side of a threshold, but in a binary or other discontinuous format (e.g., “inactive”) when the measurement is on the other side of that threshold. When truncated data is present in supervisory information used for training a computational model, there may be insufficient information to train a regression model on the truncated datapoints without additional inferential steps. For a classifier model, it becomes difficult to set thresholds for classes to fit the model.

When data is censored, a measurement may not be reported at all. In some cases, an experiment or assay may have been performed for a compound providing a measurement of bioactivity of that compound, but the measurement may not be reported. For example, the measurement may fall outside a range set by an investigator. In some cases, no experiment or assay is performed because an investigator believes the experiment or assay a-priori is unlikely to produce useful results. A large scale, untargeted, high-throughput screening program would likely have a low rate of compounds shown to have a type of bioactivity, and a large number of compounds indicated as inactive, and thus provides data that is more imbalanced. In contrast, a targeted study reported in literature would have censored data, resulting in a higher rate of compounds shown to have the type of bioactivity, compared to a large-scale screening program, and a smaller number of compounds indicated as inactive, and thus provides data that is more biased.

In general, publicly available bioactivity measurements may have a range of quality, and the quality of each source may be uncertain. Some assay protocols are more rigorously defined than others, and some assays have benefited from extensive iteration and improvement over time. Some laboratory environments are more well controlled and well equipped than others to produce repeatable and reliable measurements. Some data sources, such as the CHEMBL database, may include data that represents an attempt to assess and assign qualitative quality scores to bioactivity data.

Further, with such issues related to the data about researched items and potential candidate items, different computational models, training algorithms, training sets, and interventions to address these issues, likely will produce different results, i.e., different models and differently trained models likely will make different predictions. Typically, an “optimal” model is sought by training and testing numerous models, but often finding an optimal model is not achievable.

To address the various machine learning problems that can arise, a platform, as described herein, allows multiple machine learning experiments to be defined, and then allows predictions from those multiple machine learning experiments to be queried to provide a set of nominations. The platform can generate aggregate statistics for the predictions made over multiple machine learning experiments, and those aggregate statistics can be used to filter, sort, select, and otherwise process the set of nominations.

This use of aggregate information about predictions made by different machine learning experiments eliminates the effort of trying to find an optimal model for making predictions. Instead, multiple different machine learning experiments can be defined, using differing computational models, training sets, training algorithms, and interventions to address issues due to the data. When predicting bioactivity of compounds, by using a variety of different statistics, sorting, and filtering, the nominations are more likely to identify predicted candidate compounds having a higher likelihood of actual bioactivity if appropriate laboratory experiments are performed to verify the predicted bioactivity. This enables prioritization of further experimentation on the predicted candidate compounds.

Further, to address the various machine learning problems that can arise, a variety of techniques can be used in this platform, whether alone or in combination, for use within the multiple machine learning experiments, examples of which will be described in further detail in the following paragraphs. These techniques can be implemented within the machine learning experiments, such as in the implementations of the computational models, in the implementations of the training algorithms for these computational models, or in the selection of training sets, or in how the outputs of different computational models are evaluated whether individually, or any combination of these. The implementations of the computational models can include how features are extracted from the input data. The implementations of the training algorithms can include how supervisory information is extracted from the training set.

One technique that can be used is to use a computational model that is an ensemble of models. In such implementations, the machine learning experiment specifies a computational model that is an ensemble of multiple models. Each model in a plurality of models has a respective output. The outputs of the multiple models are input to an ensemble function, which provides a final output of the computational model. Execution of the machine learning experiment for which the computational model is an ensemble of multiple models results in a set of trained models and an ensemble function. In some implementations, parameters of the ensemble function also may be trained.

The ensemble combines outputs across models to maximize performance and generalizability. In some implementations, the ensemble is weighted so that each model contributes to a final score according to its strengths and weaknesses.

For example, as noted above, some models can make predictions based on expert-designed molecular descriptor features. These descriptors are intended to integrate prior knowledge of the feature space reflected by domain expertise, such as a deterministically computable molecular properties like charge and weight or the presence of certain subgroups known to be associated with bioactive effects. Such molecular models may have good performance in making predictions on data-poor tasks, because they require fewer examples of chemical structures to discover or learn functional structures and properties. However, they also will be stunted in their performance because their expressivity is limited to structural characteristics known a priori to have functional significance.

As another example, as noted above, some models employ graph convolutional architectures. Such graph convolutional models may better predict differences in chemical function based on complex characteristics of active substructure characteristics, but use more data to train on novel tasks, i.e., novel bioactivity types. An example of such a model is the DeepChem model identified above. Some of such models may extract functional groups as features as a result of training.

As another example, as noted above, some models incorporate edge features in molecular graphs (i.e., bonding information). Such edge feature models may better distinguish activity potential between molecules with similar chemical formulas, bulk descriptors (such as weight), or are composed of similar subgroups, yet are distinct in how they are bound together. Such models may also differentiate local and global structural interdependencies in complex ways, through message passing or similar architectures. These techniques allow the model to better represent information about large scale structures in molecules such as the interactions between multiple functional groups. An example of such a model is the ChemProp model identified above.

As another example, as noted above, some models incorporate sequence data about proteins. Such protein sequence models may better generalize to make predictions about new proteins, but are limited in their applicability beyond certain types of experimental outcomes (namely single protein binding assays and equivalents). Examples of such protein sequence models are the DeepAffinity model and the MONN model identified above.

As another example, as noted above, some models are supervised with information about the binding sites of individual atoms on proteins. Such protein binding models may better generalize across proteins even more than a protein sequence model, generating intermediate representations of molecules that are directly supervised with physical measurements of actual binding sites. However, such models are limited by the relatively small amount of detailed crystallographic data available on these binding interactions. An example of a protein binding model is the MONN model identified above.

In such implementations using an ensemble of models, the trained computational model, when applied to a selected set of potential candidate compounds, produces a result set in which each of the multiple models, and the ensemble function, provides information relevant to any prediction made for any potential candidate compound.

In such implementations, the result set comprises data representative of a set of predicted candidate compounds from among the selected set of potential candidate compounds. The information stored for each of the predicted candidate compounds can include not only a prediction value for the predicted type of bioactivity, but other data provided by the multiple models and the ensemble function.

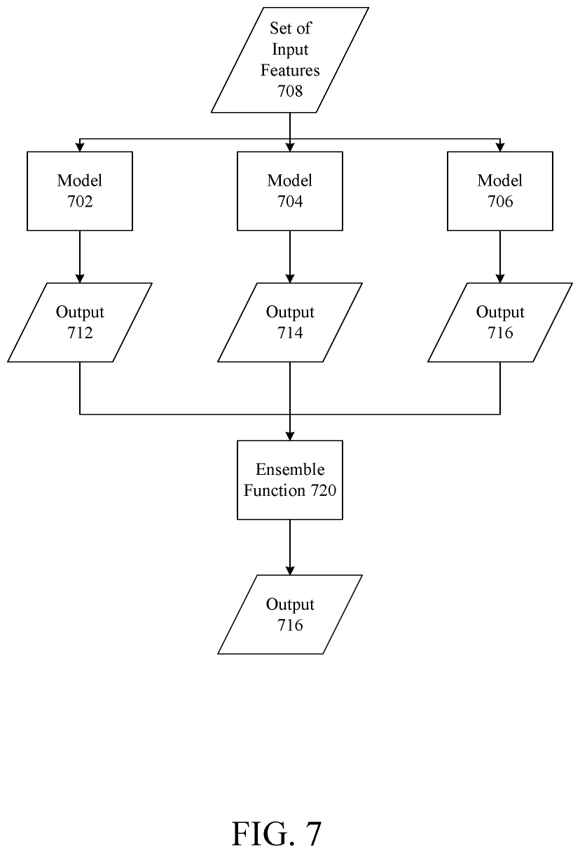

Referring now to FIG. 7, an example implementation of an ensemble of models will now be described. In this example, three models 702, 704, and 706 are illustrated, but any number of two through any positive integer N models can be used. In this example, each model receives the set 708 of input features for a given item (whether a researched compound during training, or a potential candidate compound during application of the trained model). Each model, e.g., 702, 704, 706, provides its own respective output, e.g., 712, 714, 716. An ensemble function 720 combines the outputs from the models to provide a final output 722 of the ensemble.

In some implementations, each model, e.g., 702, is trained to make predictions for the same types of bioactivity, also called “targets”. The models can be trained to make predictions for any positive integer number T of targets. Thus, after training, the output of each model is a prediction of whether a potential candidate compound has one of the T types of bioactivity, based on that model. The ensemble function combines the predictions of the multiple models to provide a final prediction value as an output. Thus, if there are M potential candidate compounds and T targets, and N models, the N models together generate M*T*N predictions. Some predictions are positive (indicating a type of bioactivity is likely); some predictions are negative (indicating a type of bioactivity is not likely); some predictions are more certain than others. Through an ensemble function (such as a weighted averaging over N), resulting in M*T final scores, some of the final scores are significant enough to assert with high confidence as positive predictions of bioactivity.

As an example implementation, a first model (e.g., 702) can implement a graph convolutional model. An example of such a model is the DeepChem model. A second model (e.g., 704) can implement a protein sequence model. An example of such a model is the DeepAffinity model. A third model (e.g., 706) can implement a protein binding model. An example of such a model is the MONN model. A fourth model (not shown) can implement an edge feature model. An example of such an edge feature model is the ChemProp model. Yet additional models can be used implementing other types of models, or models trained with differing kinds of training algorithms or supervisory information.

As noted above, the outputs of the multiple models are input to an ensemble function, which provides a final output of the computational model. There are several possible implementations of an ensemble function, of which the following are examples. The invention is not limited to the following examples.

In some implementations, a soft voting ensemble is used, which combines predictions from multiple models using a weighted average. The soft voting ensemble works by doing a weighted average of the prediction scores p_i for a given compound and target across models i=1 . . . M using a set of weights w_i. The weighted average is simply sum_i (w_i*p_i), where the weights are normalized such that sum_i (w_i)=1. The weights w_i can be determined empirically by an optimization process scored on held out training data. In some implementations, the ensemble function comprises a weighted average of the model outputs from those models that generated a prediction. In some implementations, the ensemble function comprises a weighted average of the model outputs from all models, where models that did not generate a prediction are assigned a value of zero.

In some implementations, a stacked ensemble is used, in which a second level model is trained to optimally combine the predictions of a set of independent models. In some implementations, a stacked soft voting model, which combines both approaches, can be used.

To generate weights, a variety of techniques can be used. In general, one or more factors are computed based on the individual or relative performance or training characteristics of each model. These one or more factors are processed to generate weights.

One example factor is data size. The data size is a function of the number of samples in the training set for each of the types of bioactivity. For example, training set size is known to be a powerful and direct determinant of machine learning model performance, both in the general case and specifically for cheminformatic models. Models trained on larger datasets tend to perform better than models trained on smaller datasets, although the specific characteristics of the data and the training task also play significant roles in determining performance (see, e.g., Wu, Zhenqin, et al. “MoleculeNet: a benchmark for molecular machine learning,” Chemical science 9.2 (2018): 513-530).

Another example factor is a score based on a ranking of the outputs of the models using any scoring function. An example scoring function is a normalized discounted cumulative gain (NDCG) score based on the outputs of the set of models. Such a scoring function prioritizes performance for items at the top of a ranked list, and de-prioritizes or ‘discounts’ performance for items at the bottom of the list. This procedure is highly relevant to predicted bioactivity, as the universe of predicted compounds may be far larger than the number of compounds that can be directly researched, so discriminating the potential bioactivity of a small number of compounds likely to be active is the primary actionable computational task.

Another example factor is selectivity (averaged across targets). This metric reflects how effectively the model can distinguish the activity of a compound against one target versus another, reflecting its ability to learn about the specific interactive potential of each compound and target. In general, most compounds will be selective with respect to most biological tasks; in other words, most compounds will fail to hit most targets. A model which produces a prediction which has low selectivity may therefore be less plausible than another model which does not.

Another example factor is consistency (agreement between instances of the same model). In general, machine learning models which produce highly variable predictions based on the stochastic initialization of their learning parameters or minor variations in their training data are less likely to generalize well to predictions against new compounds (see, e.g., Swabha Swayamdipta et al., “Dataset Cartography: Mapping and Diagnosing Datasets with Training Dynamics”, Oct. 15, 2020, arXiv:2009.10795v2). Models that achieve higher consistency across variations in initialization and training sets may therefore have greater plausibility.

Another example factor is agreement (agreement with the inter-model average). In a system in which models have been selected based on their known success against similar modeling tasks, it can be expected that repeated deviation from the consensus of multiple models may be an indicator of model plausibility.

Any one or more of such factors can be used, or combined, to provide weights. Given a combination of any such factors, a set of weights can be generated using another function, such as an optimization function. This is analogous to the hyperparameter optimization process used widely in machine learning. For example, a form of sequential model-based optimization (SMBO) approach can be used. SMBO methods sequentially construct models to approximate the performance modulating effect of hyperparameters based on historical measurements, and then subsequently choose new hyperparameters to test based on this model. An example of such a method includes a tree-structured Parzen estimator (TPE).

With an ensemble of models, the results from each model also allow several additional metrics to be computed and evaluated. Such metrics can be used for evaluating the individual models, or evaluating performance of the ensemble, or for sorting and filtering predicted candidate compounds.

For example, a predicted selectivity (see Table II below) can be computed for each type of bioactivity. As an example implementation of such a predicted selectivity, a formula that can be used is: 1 minus the prediction score for the type of bioactivity averaged across all compounds. The selectivity can be computed for each model in the ensemble and for the ensemble.

As another example, the results from each model, considered together, also can provide an indication of agreement among the models.

As another example, various statistics can be computed from the collection of results from the different models, such as minimum, maximum, mean, median, mode, variance, range, and so on.

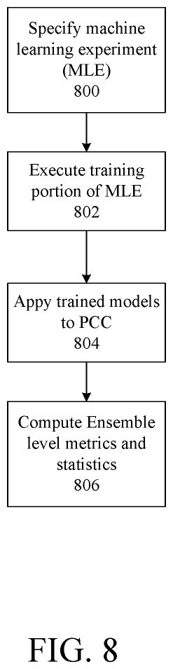

FIG. 8 is a flowchart describing operation of the ensemble. At 800, the machine learning experiment is specified. This includes specifying the training set to be used to train each of the models, each of the individual models and the ensemble function, and the set of potential candidate compounds (PCC) to which the ensemble will be applied.

At 802, the training portion of the machine learning experiment is performed. This includes independently training each of the individual models using the specified training set.

At 804, the trained models are then applied to the set of potential candidate compounds. Each trained model outputs a prediction value for each potential candidate compound. These values can be stored in the database table 214.

At 806, the ensemble level score for each potential candidate compound is computed using the ensemble function. This ensemble level score can be stored in the database table 214. Other statistics related to the ensemble also can be computed per compound.

Another technique that can be used is to incorporate uncertainty modeling into a computational model. Uncertainty modeling relates to discounting predicted activity of a primary model by, for example, predictions of a secondary model or through specialized post-processing of the predictions of the primary model. The secondary model can be any uncertainty model that can assess the reliability of the primary model. As an example, an uncertainty model can assess differential reliability of a deep neural network (DNN). As another example, an uncertainty model can be an analytical approximation of the uncertainty of the primary model.

An uncertainty model can be in itself a computational model that outputs its own prediction value. The input features for the uncertainty model can be derived in several ways, such as one or more of the following techniques. For example, the input features can be generated using various embedding techniques, such as autoencoders or other transforms, based on the data about the items processed by the primary model. The input features may include the output predictions of the primary model. The input features can include all or a subset of the input features of the primary model. Herein the prediction value output by the uncertainty model is called the “uncertainty value” to distinguish it from the prediction value output by the primary model of which reliability is being assessed. In some implementations it is desirable to assess the suitability of the uncertainty model.

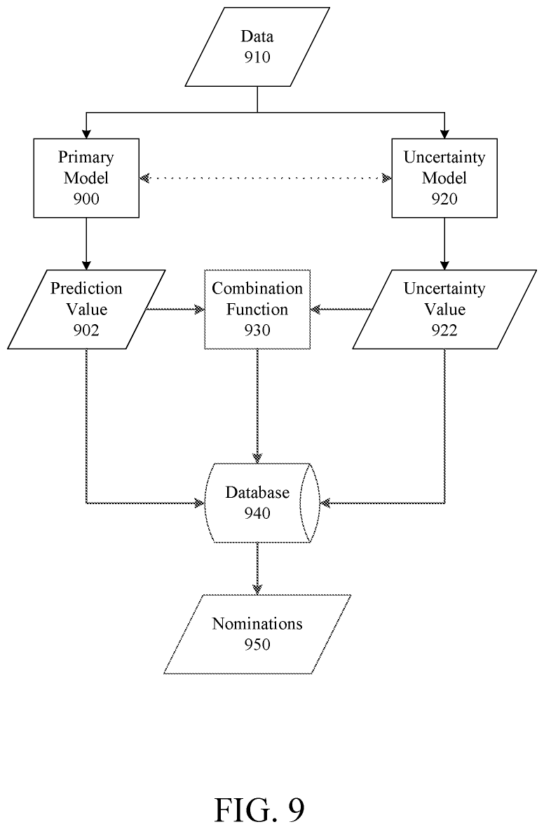

Thus, as shown in FIG. 9, in an example implementation of a computational model incorporating uncertainty modeling, a primary model 900 is the computation model specified in the machine learning experiment that generates the primary prediction values 902 for compounds for the types of bioactivity, using data 910 (the illustration in FIG. 9 assumes the primary model and uncertainty model have been trained). For each prediction for a compound-type of bioactivity pair, the uncertainty model 920 generates an uncertainty value 922. The uncertainty value also can be stored in the database 940 of results (e.g., table 204 in FIG. 2) along with the prediction value 902. A combination function 930 implements one or more functions that combine the prediction value and the uncertainty value, examples of which are described in more detail below, and the result of this also can be stored in the database 940 or computed in real time when requested. The information about the uncertainty value and a result of the combination function can be used in the nominations 950, as described in more detail below. One or more combination functions can be used, and storage of the prediction value and uncertainty value in the database allows different combination functions to be applied at different times and for different purposes.

Examples of such an uncertainty model include, but are not limited to, the following. One or more uncertainty models, including models if different types, can be used in combination.

A residual model is a model that is trained to predict primary model residuals on held out data. “Residuals” are quantitative differences between the predicted score of the primary model and the ground truth supervising label of the training data. An example of such a model implementation is described in Hie, Brian, Bryan D. Bryson, and Bonnie Berger. “Learning with uncertainty for biological discovery and design.” BioRxiv (2020)). An example of a residual model function is a Gaussian process model.

A self-supervision-based model is a model that is trained to predict primary model residuals based on performance against one or more auxiliary tasks. While some systems use self-supervising auxiliary tasks to augment supervised learning performance, an uncertainty model based on self-supervision involves training an uncertainty model based on the self-supervising tasks.

A deep ensemble-based model measures variance across an ensemble of primary models, each of which is trained with different random seeds and data subsets. An example of a model is described in “Simple and Scalable Predictive Uncertainty Estimation,” by Ralaji Lakshminarayanan, et al., available at arXiv:1612.01474v3. Another example of a model is described in “Evaluating Scalable Uncertainty Estimation Methods for DNN-based Molecular Property Prediction,” by Gabriele Scalia et al., and available at arXiv:1910.03127.

In some implementations of the residual model, features of a compound to be input to the model can be generated by featurizing the chemical structure of the compound. For example, an autoencoder referred to as “CDDD” can be used, which is described in Winter, Robin, et al. “Learning continuous and data-driven molecular descriptors by translating equivalent chemical representations.” Chemical science 10.6 (2019): 1692-1701. For example, an autoencoder referred to as “Junction Tree” can be used, which is described in Jin, Wengong, Regina Barzilay, and Tommi Jaakkola. “Junction tree variational auto encoder for molecular graph generation.” arXiv preprint arXiv:1802.04364 (2018). The Gaussian process model is itself a computational model that outputs an uncertainty value. The uncertainty value output by the GP model (GP (G)) is fit to predict the residual of a deep learning model (D) as a function of a chemical embedding (X) on an activity task (y): GP(x)˜y˜D(x). The suitability of a Gaussian process model can be assessed by its ability to improve ranking performance, which can be measured using a score such as a normalized discounted cumulative gain (NDCG).

In some implementations of a self-supervision-based uncertainty model, features of a compound to be input to the model can be derived using “auxiliary tasks,” such as deterministically calculated molecular descriptors. Such descriptors include, but are not limited to, properties such as weight, solubility, shape, or any other molecular property that can be computed through means independent of the primary machine learning model. As an example, any descriptor computed by the cheminformatics software package RDKIT can be used. The auxiliary tasks are a form of data augmentation that enriches a training set by incorporating external data, metadata, or computed data. The self-supervision-based model is itself a computational model, such as a random forest-based model, that outputs an uncertainty value.

In some implementations, the self-supervision-based model can be trained on residuals measured for the auxiliary tasks. In effect, the model learns a map between the molecular features of the input compounds and the reliability of the primary model. For example, the model could learn that molecules that include or do not include a certain functional group typically have a larger or smaller error in the primary model predictions. Suitability of the self-supervision-based uncertainty model can be assessed by its predictive performance on the residuals of the primary task when trained using features based on the residuals for the auxiliary tasks for held out data, meaning that training data is used to directly assess the accuracy of the self-supervision-based model in predicting the error of the primary model. A learner (G) is fit to predict the residual of a multi-task deep learning model (D) as a function of a chemical and/or bioactivity task embedding (X) on a set of auxiliary tasks (t) learned in parallel to the primary activity tasks (y): G(x, t)˜y˜D(x, t). The auxiliary tasks are chosen to be molecular descriptors (like weight) that strongly distinguish the test and target domains.

The auxiliary tasks have additional benefits. In neural network models, the additional auxiliary tasks improve neural representation layers learned during model training which can improve performance on the primary tasks. Also, they improve generalization of the trained model to new compounds. Also, neural networks can be pretrained on a larger dataset to initialize how the model represents chemical properties.

In such implementations, both the uncertainty value(s) for a compound and a type of bioactivity as output by the uncertainty model(s) and the prediction value for the compound and the type of bioactivity as output of the computational model can be used to evaluate predicted candidate compounds.

For example, the uncertainty value for a compound and a type of bioactivity as output by the uncertainty model can be combined with the prediction value for the compound and the type of bioactivity as output of the computational model. In some implementations, a function based on a sum of the uncertainty value and the prediction value can be computed, effectively representing an upper confidence bound. In some implementations, a function based on subtracting the uncertainty value from the prediction value can be computed, effectively representing a lower confidence bound. Weights can be applied to uncertainty values in such functions. In ensembles of primary models, multiple independent uncertainty estimates also can be used in combination.

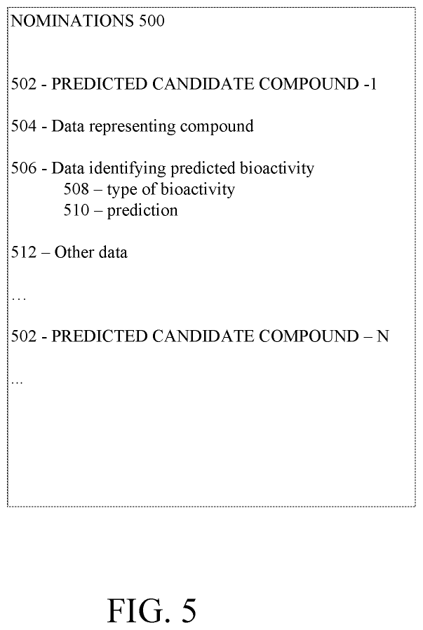

As described in more detail below, uncertainty values, or other data computed based on uncertainty values, can be included in nominations 950 (e.g., see 500 in FIG. 5) of predicted candidate compounds, and can be used to sort and filter such nominations.

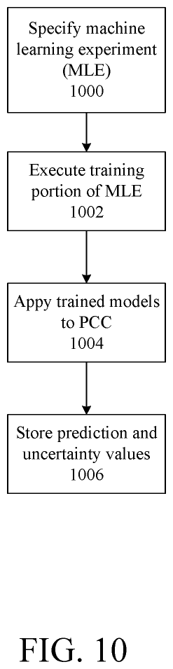

FIG. 10 is a flowchart describing operation of the computational modeling incorporating uncertainty modeling.

At 1000, the machine learning experiment is specified. This includes specifying the training set to be used to train the computational model, specifics of the computational model, and the set of potential candidate compounds to which the trained computational model will be applied. The specification of the computational model can include a specification of the primary model and the uncertainty model. There can be more than one uncertainty model. The combination function used to combine the prediction values and the uncertainty values can be included in, or separate from, the specification of the machine learning experiment.

At 1002, the training portion of the machine learning experiment is performed. This includes training the primary model using the specified training set and training the uncertainty model using the specified training set along with any auxiliary tasks, embeddings, autoencoders, or augmented data.

At 1004, the trained models, both the primary model and the uncertainty model(s), are then applied to the set of potential candidate compounds. The trained primary model outputs a prediction value for each potential candidate compound and type of bioactivity. The trained uncertainty model outputs an uncertainty value for each potential candidate compound and type of bioactivity. These values can be stored 1006 in the database table 214. In some implementations, a combination of the prediction value and the uncertainty value can be used to determine whether the compound should be identified as a predicted candidate compound for which the values should be stored in the database.

Another technique that can be used is to incorporate sample weighting into a computational model. Sample weighting addresses the problem of domain shift. Sample weighting involves upweighting samples close to the target domain during training. Class imbalance is addressed by equalizing the class weight of the training samples. Metrics used for sample weighting also can be reported for predicted items to help filter and sort predicted items.

In some implementations, one technique that can be used to compute sample weights is to compute respective distance metrics or similarity metrics between potential candidate items and researched items in the training set. The computed metric can be any of a variety of distance metrics or similarity metrics depending on the nature of the items. The weight for any given item in the training set can be computed as a function of one or more computed metrics. For example, the weight of a sample can be a function of the inverse of how close the sample is to the target domain. The closeness of a sample to a target domain can be a function of the distance/similarity of the sample to its positive integer number N nearest neighbors in the target domain. Multiple different weights can be computed using different functions.

In some implementations, the computed metric is used to identify researched items in the training set that are most similar to potential candidate items in the target set. In some implementations, training can be performed using only those identified researched items most similar to potential candidate items in the target set. In some implementations, the researched items identified as most similar to potential candidate items in the target set can be weighted for training.

In some implementations, the weights of training samples can be equalized among the classes to address class imbalance, such as by using a technique described in Kouw, Wouter M., and Marco Loog, “An introduction to domain adaptation and transfer learning,” arXiv preprint arXiv:1812.11806 (2018).

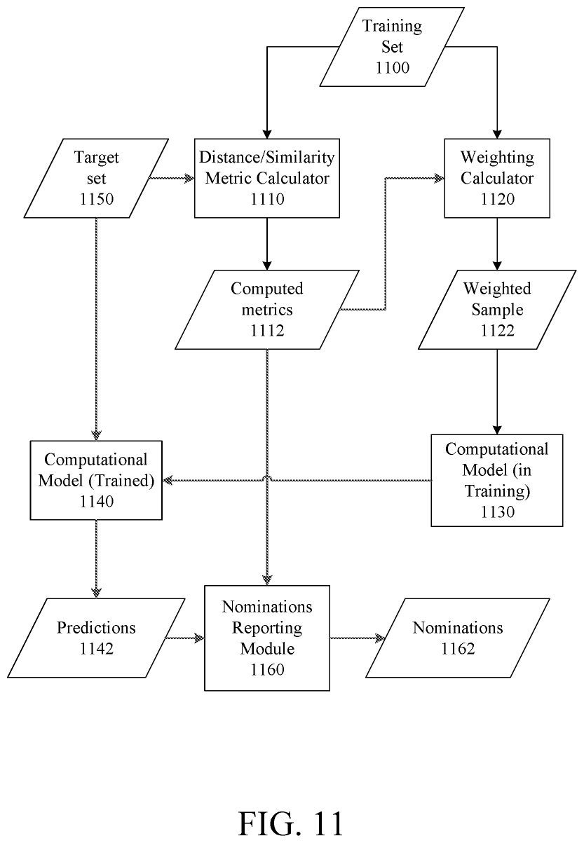

Referring now to FIG. 11, an example implementation of a computational model incorporating sample weighting will now be described. In this example, samples from a training set 1100 and a target set 1150 are inputs to a distance metric or similarity metric calculator 1110. One or more computed metric(s) 1112 is generated for each pair of training and target sample input to the calculator 1110. The computed metrics 1112 are an input to a weighting calculator 1120. The weighting calculator computes a weight for a sample in the training set 1100 based on its corresponding computed metric(s) 1112, weights the sample accordingly, and outputs the weighted sample 1122. The weighted samples 1122 from the training set are inputs to the computational model 1130 during the training process.

After the model is trained, the trained model 1140 receives samples from the target set 1150 to make predictions 1142. For items identified as predicted candidate items, the computed metrics 1112 with respect to the training samples can be stored as corresponding to the target items. These metrics provide a measure of the domain shift between a predicted candidate and samples in the training set. When nominations 1162 about predicted candidate items are presented, such as through a nominations reporting module 1160, such computed metrics or other data computed based on such metrics can be included in the nomination information (see 500 in FIG. 5).

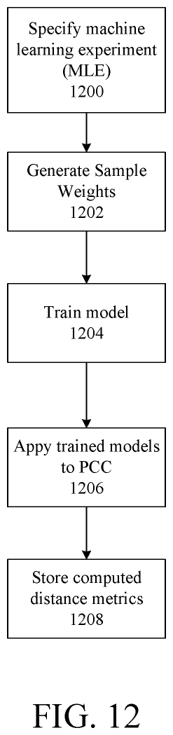

FIG. 12 is a flowchart describing operation of the computational modeling incorporating sample weighting.

At 1200, the machine learning experiment is specified. This includes specifying the training set to be used to train the computational model, specifics of the computational model, and the set of potential candidate compounds to which the trained computational model will be applied. The specification of the computational model can include a specification of any sample weighting to be used. There can be more than one kind of sample weighting and more than one computational model. The distance metric or similarity metric used to compare samples from the training set to samples in the set of potential candidate compounds can be included in, or separate from, the specification of the machine learning experiment. For example, the distance metrics, corresponding weights, and even the weighted training samples, can be precomputed from the perspective of executing a specified machine learning experiment. That is, a set of computed weights or weighted samples can be data used by the machine learning experiment.

At 1202, the sample weights for the training set are generated. As noted above, the distance or similarity metrics, and resulting weights, can be precomputed with respect to training the computational model. Or, the machine learning experiment can be specified to include a description of how the metrics and weights are to be computed and applied.

At 1204, the training portion of the machine learning experiment is performed. This includes training the computational model using the weighted samples from the training set.

At 1206, the trained models are applied to the set of potential candidate compounds. The trained primary model outputs a prediction value for each potential candidate compound and type of bioactivity. The prediction values can be stored in the database (e.g., data structure 214 in FIG. 2).

The computed distance metric(s), or function(s) of them, also can be stored (1208) along with the prediction values for each predicted candidate compound. Such data can be treated as statistics that describe the domain shift between the training set and the target set.

Some example distance metrics that can be used between researched compounds and potential candidate compounds include, but are not limited to, the following. Any one or more of these or yet other metrics, can be used.

A Tanimoto metric can be used. The Tanimoto coefficient, also called the Jaccard index, is a measure of set similarity generally applied to binarized data. With respect to chemical compounds, molecular fingerprints are generated through techniques such as the Morgan algorithm. The binary features of these fingerprints reflect the presence or absence of certain chemical fragments in a molecule. The Tanimoto coefficient between two fingerprints can then be calculated to measure the distance between two compounds with respect to the shared presence or absence of these fragments.

A molecular feature distance metric can be used. This metric can be calculated as a Euclidean distance, or other distance metric, between two compounds in any feature space, such as the space of molecular descriptors as defined herein. The Euclidian distance calculation, or other distance metric, over these features implicitly weights all the features equivalently. This approach is advantageous in its simplicity, but may introduce bias related to the collinearity or inter-dependence of multiple molecular descriptors.

A reduced dimensionality projection distance can be used associated with a dimensionality reduction algorithm such as Principal Component Analysis (PCA). This approach extends the molecular feature distance by computing the distance on a transformed feature space, rather than calculating a Euclidian distance. Transformations such as the PCA dimensionality reduction algorithm are beneficial because they project the molecular feature space into a lower dimensional plane which helps to alleviate collinearity between features and helps to privilege features based on their ability to explain differences between compounds within the dataset.

Another kind of distance metric is based on a class of techniques called deep neural embeddings. Such embeddings provide ways to featurize the chemical structure of the compound. For example, an autoencoder referred to as “CDDD” can be used, which is described in Winter, Robin, et al. “Learning continuous and data-driven molecular descriptors by translating equivalent chemical representations.” Chemical science 10.6 (2019): 1692-1701. For example, an autoencoder referred to as “Junction Tree” can be used, which is described in Jin, Wengong, Regina Barzilay, and Tommi Jaakkola. “Junction tree variational auto encoder for molecular graph generation.” arXiv preprint arXiv:1802.04364 (2018). These techniques provide another way to convert a symbolic representation of a molecule, such as a SMILES string, into a mathematical vector. Distances can then be computed between different vectors of different molecules. These techniques have a potential advantage over molecular fingerprint-based embeddings in that they can be generated using machine learning models, so they can potentially learn significant molecular structural features that are not captured by the rule set associated with a fingerprint algorithm.

After machine learning experiments have been run, potential candidate compounds may have become identified as predicted candidate compounds with respect to one or more types of bioactivity. As noted above in connection with FIG. 2, the data for a predicted candidate compound may include one or more respective types of bioactivity associated with that compound, and a respective prediction value for each type of bioactivity associated with that compound. The database can be queried, for example by using one or more types of bioactivity, to access data about predicted candidate compounds and their respective prediction values. For example, one or more values for the task identifier field 272 can be used to identify all predictions made for the corresponding one or more types of bioactivity. Other information about the predicted candidate compounds can be accessed as well. For example, as shown with the aggregation processor 140 in FIG. 1, various statistics can be computed from the collection of result sets from multiple machine learning experiments. A user interface, such as the second interface 130 in FIG. 1, can be used by an end user to input a query and to view results of the query.

A variety of queries can be generated to access such data. For some purposes, one may identify predicted candidate compounds for a selected type or types of bioactivity (e.g., the value in the task identifier field 272 matches one or more selected values), and where the prediction value is above a threshold (e.g., the value in field 274 is above a threshold).

For some purposes, one may identify predicted candidate compounds based solely on their prediction value (e.g., the value in field 274 is above a threshold, which may be selected to be particularly high), regardless of the type of bioactivity. Such as query is helpful, for example, to identify likely candidates for experimental verification regardless of bioactivity type.

For some purposes, one may identify predicted candidate compounds for a selected type or types of bioactivity (e.g., the value in the task identifier field 272 of a first entry matches one or more selected values), but which do not have another bioactivity or are not predicted to have that other bioactivity (e.g., where the predicted candidate compound identified by the compound identifier field 270 in the first entry does not have another entry in either the prediction table 204 or the bioactivity table 214 where the task identifier 272 or 252 matches some other selected one or more values). Such a query is helpful, as an example, where the other bioactivity may be associated with a side effect.

For some purposes, one may identify predicted candidate compounds for multiple selected types of bioactivity (e.g., the value in the task identifier field 272 matches multiple selected values), where those types of bioactivity are related or similar. For example, the types of bioactivity may be interactions with a set of proteins associated with a biological pathway.

For some purposes, one may identify, for each predicted candidate compound, the various types of bioactivity the system as predicted (e.g., for each compound, a list of all the values in the task identifier field 272 from respective entries for the compound). This list may be augmented by types of bioactivity already known for the compound as well by querying the bioactivity table. Such a query is helpful to then use the lists to identify compounds having similar bioactivity profiles as each other, which therefore may have either synergistic or competitive effects.

The accessed data, or nominations, can be processed and presented to a user as a set of ranked, filtered, or sorted (or any combination of these) predictions. Using such predictions, actual laboratory experiments can be performed to assess or verify whether the predictions are accurate. The results from such actual laboratory experiments can be stored in the database and used for future predictions.

An example of a format for data returned from a query is shown in FIG. 5. In FIG. 5, a nominations table 500 stores a row 502 for each compound predicted to have a type of bioactivity, with any positive integer number N of rows. Note that, if multiple machine learning experiments resulted in multiple predictions that a compound has a type of bioactivity, a separate row 502 can be generated for each prediction. In this example, for each predicted compound, there can be data 504 representing the compound itself, such as an identifier (e.g., the value from field 270 of the prediction table) of the compound (e.g., an InChiKey identifier, or SMILES string, or others), a common name for the compound, and whether and where the compound is commercially available for purchase. For each predicted compound, there also can be data 506 identifying the predicted bioactivity, such as data 508 identifying the type of bioactivity (e.g., the value from the task identifier field 272 of the prediction table) and data 510 about the prediction, such as the prediction value 274.

A wide variety of other information 512 about the predicted compound and the prediction can be used in queries, or provided in the nominations table 500, or used for sorting or filtering in the user interface, or any combination of these. Specifically, such information can include various statistics aggregated or computed across several result sets from multiple machine learning experiments, or metadata about the compounds, or other information resulting from transformations of stored metadata about the predicted candidate compounds, or any combination of these. Some examples of aggregate or other data are described in the following.

For any predicted candidate compound, such a query enables several aggregate statistics to be computed about the predicted candidate compound. The system can compute a function based on a number of machine learning experiments that predicted this predicted candidate compound to have this type of bioactivity. The system can compute a function based on a number of types of bioactivity that a compound is predicted to have. The system can compute a function, such as a sum or average, based on the prediction values for the types of bioactivity that a compound is predicted to have. Any one or more of these, and yet other statistics, can be computed from the database result sets.

For any type of bioactivity, such a query also enables several aggregate statistics to be computed about the type of bioactivity. The system can compute a function based on the compounds predicted to have this type of bioactivity, such as the number of compounds predicted to have this type of bioactivity. The system can compute a function based on the prediction values for the compounds predicted to have this type of bioactivity, such as the average prediction value across the compounds predicted to have this type of bioactivity. Any one or more of these, and yet other statistics, can be computed from the database result sets, and may be computed in combination about statistics computed about predicted candidate compounds.

Examples of data that can be included in the nominations 500 include, but are not limited to, information such as shown in Table I below:

TABLE I

| Term | Description | Source |
|---|---|---|
| Protein | The selectivity of the protein across the results sets for | Aggregation over |
| selectivity | one or more multiple learning experiments. Example: | predictions from |
| (Predicted) | 1 minus the prediction score for the protein averaged | table 214 |
| | across all compounds. | |
| Protein | The selectivity of the protein according to ground | Aggregation over |
| selectivity | truth data from a database, such as the ChEMBL | training data from |
| (Ground Truth) | database or the Tox21 database, calculated as 1 minus | Table 212 |
| | the average of the ground truth inhibition label for all | |
| | compounds with data against each protein. | |
| Number of | Number of machine learning experiments reporting a | Aggregation over |
| predictions | prediction for this compound and type of bioactivity. | predictions from |
| | | table 214 |
| Compound | Related to total number of types of bioactivity that this | Aggregation over |
| promiscuity | compound is predicted to have. Example: sum of | predictions from |
| (Predicted) | prediction scores for this compound over all predicted | table 214 |
| | bioactivity types. Typically larger when a compound | |
| | has more predicted bioactivity types. | |
| Compound | The selectivity of the compound, calculated as 1 | Aggregation over |
| selectivity | minus the prediction score for the compound averaged | predictions from |
| (Predicted) | across all predicted bioactivity types. Typically larger | table 214 |
| | when a compound has fewer predicted bioactivity | |
| | types. | |
| Assay Identifier | Information identifying any assay that can be used to | Metadata from |
| or Assay Name | verify the predicted bioactivity. | Table 216 |
| Mode | Where applicable, any mode related to the bioactivity. | Metadata from |
| | Example: an assay may be run to identify actives that | Table 216 |
| | are either an agonist or an antagonist. | |

When using an ensemble of models, data that can be included in the nominations 500 can include additional information for a compound, related to the ensemble, and models within that ensemble, that made a prediction for that compound for a type of bioactivity. Examples of such data are shown in Table II below:

TABLE II

| Term | Description | Source |
|---|---|---|
| Bioactivity- | The difference between the weighted | Aggregation over |
| adjusted WCP | conservative probability (WCP) of the | predictions from |
| within the | compound-bioactivity pair versus the | table 204 |
| ensemble | bioactivity-level average. Positive values | |
| | indicate the predicted association between the | |
| | pair is greater than the average compound for | |
| | that bioactivity. | |
| Weighted | The voting ensemble score (weighted average | Aggregation over |
| conservative | of the model outputs), including even those | predictions from |
| probability (WCP) | models that did not generate a prediction for | table 204 |
| within the | the compound for this type of bioactivity. The | |
| ensemble | missing models are replaced by 0, effectively | |
| | down-weighting the prediction. | |
| Weighted | The voting ensemble score (weighted average | Aggregation over |
| probability within | of the model outputs), including only models | predictions from |
| the ensemble | that generated a prediction for the compound | table 214 |
| | for this bioactivity. | |
| Predicted | The selectivity of a type of bioactivity, such as | Aggregation over |
| selectivity of a type | a protein, according to the model ensemble. | predictions from |
| of bioactivity | Can be calculated as 1 minus the average | table 214 |
| within an | prediction score across compounds for that | |
| ensemble | type of bioactivity. | |
| Size of training set | The number of researched compounds for | Processing data from |
| for the ensemble | which information characterizing the type of | database table 202 or |
| | bioactivity also can be determined, to provide | data specifying the |
| | an indication of the number of positive and | machine learning |
| | negative examples for training with respect to | experiment. |
| | that bioactivity. | |
| Number of | The number of models in an ensemble that | Aggregation over |
| Predictions in | predict that a compound has a type of | predictions from |
| Ensemble | bioactivity | table 214. |

When using a computational model that incorporates uncertainty modeling, additional data can be included in the nominations 500 related to such uncertainty modeling, such as shown in Table III below. As an example, nominations can be augmented by rank ordering on the sum of the prediction value output by the computational model for a compound and an uncertainty value output for the compound by the uncertainty model. In such rankings, an upper confidence bound (UCB) metric can be used.

TABLE III

| Term | Description | Source |
|---|---|---|
| Estimated | The confidence/uncertainty in the prediction for this | Predictions |
| confidence/ | compound - bioactivity pair as estimated by the Gaussian | from table |
| uncertainty | Process-based uncertainty model. The confidence values are | 214 |
| value | based on uncertainty predictions from the GP residual | |
| (GP) | regression model based on compound and bioactivity target | |
| | embeddings. In one implementation, the uncertainty | |
| | predictions can be inverted, squared (to represent inverse | |
| | variance), and then scaled to a mean of 1, thus providing a | |
| | normalized confidence value with a baseline of 1. | |
| Estimated | The confidence/uncertainty in the prediction for this | Predictions |
| confidence/ | compound-bioactivity pair as estimated by the Self | from table |
| uncertainty | Supervision-based uncertainty model. The confidence | 214 |
| value | values are based on uncertainty predictions from the SS | |
| (SS) | classifier regression model based on predictive performance | |
| | on molecular descriptors. In one implementation, the | |
| | uncertainty predictions can be inverted, squared (to | |
| | represent inverse variance), and then scaled to a mean of 1, | |
| | thus providing a normalized confidence value with a | |
| | baseline of 1. | |
| Estimated | The confidence/uncertainty in the prediction for this | Predictions |
| confidence/ | compound-bioactivity pair as estimated by the Deep | from table |
| uncertainty | Ensemble-based uncertainty model. In one implementation, | 214 |
| value | the uncertainty predictions can be inverted, squared (to | |
| (DE) | represent inverse variance), and then scaled to a mean of 1, | |
| | thus providing a normalized confidence value with a | |
| | baseline of 1. | |
| Combined | Any value computed as a combination of the uncertainty | Predictions |
| Uncertainty | value and prediction value for this compound-bioactivity | from table |
| and Prediction | pair | 214 |

When using a computational model that incorporates sample weighting, additional data can be included in the nominations 500 related to such sample weighting, such as shown in Table IV below. The examples below are described based on N-nearest neighbors, where N is 20. N can be any positive integer number. This data generally provides an indication of the domain shift between the source domain of the training set and the target domain (the potential candidate compounds). In some implementations, the computed distance and similarity metrics can be used to identify the N-nearest compounds to a given compound, and information about such compounds also can be provided in the nominations.

TABLE IV

| Term | Description | Source |
|---|---|---|
| mean_distance_PCA_k_20 | The mean molecular distance | Transformation of |
| | between this food compound and its | metadata from |
| | 20 nearest neighbors in the training | Table 200 |
| | dataset, as measured from a PCA | |
| | projection of molecular descriptor | |
| | features. | |
| min_distance_PCA_k_20 | The minimum molecular distance | Transformation of |
| | between this food compound and its | metadata from |
| | 20 nearest neighbors in the training | Table 200 |
| | dataset, as measured from a PCA | |
| | projection of molecular descriptor | |
| | features. | |
| max_distance_PCA_k_20 | The maximum molecular distance | Transformation of |
| | between this food compound and its | metadata from |
| | 20 nearest neighbors in the training | Table 200 |
| | dataset, as measured from a PCA | |
| | projection of molecular descriptor | |
| | features. | |
| mean_distance_Mol_k_20 | The mean distance between this | Transformation of |
| | food compound and its 20 nearest | metadata from |
| | neighbors in the training dataset as | Table 200 |
| | measured by Euclidean distance | |
| | across unreduced molecular features. | |
| min_distance_Mol_k_20 | The minimum distance between this | Transformation of |
| | food compound and its 20 nearest | metadata from |
| | neighbors in the training dataset as | Table 200 |
| | measured by Euclidean distance | |
| | across unreduced molecular features. | |
| max_distance_Mol_k_20 | The maximum distance between this | Transformation of |
| | food compound and its 20 nearest | metadata from |
| | neighbors in the training dataset as | Table 200 |
| | measured by Euclidean distance | |
| | across unreduced molecular features | |
| mean_distance_Tanimoto_k_20 | The mean distance between this | Transformation of |
| | food compound and its 20 nearest | metadata from |
| | neighbors in the training dataset as | Table 200 |
| | measured by a Tanimoto distance | |
| | over molecular fingerprints. | |
| min_distance_Tanimoto_k_20 | The minimum distance between this | Transformation of |
| | food compound and its 20 nearest | metadata from |
| | neighbors in the training dataset as | Table 200 |
| | measured by a Tanimoto distance | |
| | over molecular fingerprints. | |
| max_distance_Tanimoto_k_20 | The maximum distance between this | Transformation of |
| | food compound and its 20 nearest | metadata from |
| | neighbors in the training dataset as | Table 200 |
| | measured by a Tanimoto distance | |
| | over molecular fingerprints. | |

In the context of using information about researched compounds to identify predicted candidate compounds, which are predicted to have some bioactivity, given another a set of potential candidate compounds, there are several use cases for this kind of machine learning platform.

In some applications, the researched compounds include mostly small molecules of drugs and pharmaceuticals, and the potential candidate compounds are molecules found in foods and food products, whether naturally occurring or not, especially any compounds that are generally recognized as safe (GRAS). Data about these two sets of compounds could be used, for example, to: identify food compounds that have similar bioactivity as drugs; identify food compounds that enhance bioactivity of drugs; identify food compounds that interfere with bioactivity of drugs; or identify combinations of such food compounds.

Some applications relate to identifying compounds that are predicted to have a type of bioactivity that relates to activity of a drug. For example, there may be compounds, whether synthetic or naturally occurring, which may interfere with, or enhance, activity of a drug. Given the relationship between certain compounds and foods, the predictions can result in an indication of certain foods that contain compounds that affect the activity of the drug.

Some applications relate to identifying compounds that are predicted to have a type of bioactivity that is similar to the activity of a drug. For example, there may be compounds, whether synthetic or naturally occurring, which may provide a similar activity as a drug, allowing replacement of that drug with another compound.

Some applications relate to identifying bioactivity of a set of compounds, and in turn any impact that such bioactivity would have on health. For example, the set of compounds may be present separately, from multiple sources, or present together in a single source. The multiple sources could be multiple administered products, such as pills or liquids, or multiple food sources (whether naturally occurring, processed, or manufactured), or a combination of one or more administered products and one or more food sources.

Some applications relate to creating a set of compounds based on a disease profile such that the set of compounds together works to counteract effects of the disease. The bioactivity of compounds can be predicted and verified to develop the set.

Some applications relate to analyzing a known composition or set of compounds (e.g., a food or beverage) for associations with a disease or health state. The compounds in the food or beverage or other composition can be evaluated to predict, then verify, their bioactivity, with the goal of identifying compounds in the composition which are most likely to cause the effects observed, either positive or negative.

Some applications relate to evaluate what a person consuming, such as their diet. The evaluation can relate to, for example, what the person can expect in terms of health effects, positive or negative, and help them optimize these health effects. The compounds in their diet can be evaluated to predict, then verify, their bioactivity, with the goal of identifying compounds in the composition which are most likely to cause the effects observed.

Some applications relate to identifying compounds that are predicted to have a type of bioactivity that may result in adverse health effects. For example, there may be compounds, whether synthetic or naturally occurring, which may be toxic, carcinogenic, or have other adverse effects. Given a set of compounds known to be present in or on a product that may become present in or on a living thing, predictions about bioactivity of these compounds can be made. For example, compounds in agricultural use, such as pesticides, fungicides, fertilizer, or irrigation, or food production, handling, packaging, or distribution, can be screened for potential bioactivity.

By making such predictions, laboratory experiments can be performed to validate the predictions, such as performing an assay with a candidate compound and a selected protein to characterize the interaction of the candidate compound and the selected protein. Interaction information for a plurality of compounds can be aggregated. This aggregated information can be used to characterize an overall effect of the plurality of compounds with respect to a health condition or activity of a drug.

The results generated from multiple machine learning experiments can be provided to many kinds of users for many purposes. For example, a manufacturer of a food product can identify whether compounds in the food product may have previously unknown bioactivity. For example, a manufacturer of a drug or other small molecule can identify whether compounds in foods or other products may have potential interactions. Researchers may identify compounds predicted to have types of bioactivity with the goal of developing and performing laboratory experiments to verify such predictions. An interface can be provided for known bioactivity of any compound to be submitted to the database of researched compounds. Some applications can focus on accessing the research compounds after bioactivity of predicted candidate compounds has been verified.

Some additional examples of ways in which such a system can be used include, but are not limited to, the following.

The system can be used to develop physical products for consumption.

For example, beneficial food compounds can be identified which can deliver drug-like positive effects acting on the same primary target as a known drug. As another example, food compounds that act as drug adjuvants can be identified. These compounds deliver effects that synergize with drug activity by acting on additional proteins in the targeted pathway. As another example, food compounds that modulate side effects of drugs can be identified. These compounds are capable of cancelling or reinforcing drug activity on a primary or bystander pathway to relieve the experience of side effects.

The system also can be used to deliver digital content, to both consumers and businesses.

As one example, a web-based consumer user interface can be provided to allow an individual to input food products from a diet, drugs or other therapies being taken, or both. The system can be used to identify potentially harmful food-drug interactions, because the system can identify food compounds that have negative effects which interfere with drug activity. The system also can be used to aggregate profiles of food compounds, or foods that include a number of known compounds. The system can be used to combine one or more effects of nominated compounds to characterize the overall effect of a food compound or composite product against a targeted drug or disease.

As another example, the system can provide information that nominates compounds for laboratory validation, where the nominated compounds are any compounds identified in the other use cases where the effect of the compound is predicted, but not verified. The system also can be used in a form of active learning loop. In such a configuration, the system can be used to recommend compounds for laboratory experimentation on the basis of both their potential product value and also their potential to influence future training iteration of the machine learning models used in the system.

Using a system such as described herein, given information about an individual, such as a patient, data about items relevant to that patient also can be identified.

For example, patient medical history information, such as patient conditions, can be processed to identify drugs or other compounds known to be used for treatment for the patient's conditions. The database of predicted candidate compounds (or researched compounds) can be searched for other compounds, such as compounds found in foods, which have similar effects as the drugs or other compounds known to be used for treatment for the patient's conditions. These food compounds, and the foods containing them, can be proposed to the patient as a dietary change or supplement.

As another example, patient diet information can be processed to identify food compounds known to be present in those foods or to identify molecular intake patterns. The database of predicted candidate compounds (or researched compounds) can be searched for whether those compounds are known to have, or are predicted to have, similar effects as or interactions with drugs. The existence of known positive or negative effects between diet and drugs could provide information related to clinical trials, such as whether a patient can qualify for a clinical trial, or to explain results from a clinical trial.

Accordingly, in any of the following aspects, a computational model can include an ensemble of models. Computed statistics can include data describing how outputs of the multiple models in the ensemble are combined.

In any of the following aspects, a computational model can include a primary model and an uncertainty model. The primary model and the uncertainty model are trained. The trained primary model and the trained uncertainty model are applied to data representing potential candidate items. For a predicted candidate item, the trained uncertainty model provides an uncertainty value for the item. Statistics that can be computed and used for prioritizing items can be based on the uncertainty values for items. An uncertainty model can be provided for an ensemble of models or for each model within an ensemble or both.

In any of the following aspects, weights for the selected subset of the plurality of researched items can be computed based on a distance metric or similarity metric between the researched items and the selected subset of potential candidate items. The computational model can be trained using the weighted data representing the plurality of researched items. The computed statistics for predicted candidate compounds can be based on the distance metric or similarity metric between the researched compounds and the predicted candidate compounds.

In one aspect, a machine learning system trains computational models using data representing a subset of researched items and applies the trained computational models to data representing a subset of potential candidate items to provide result sets for the computational models. A result set includes data representative of a set of predicted candidate items from among the potential candidate items. A computational model outputs, for each predicted candidate item, respective predicted information for a property of the predicted candidate item. The machine learning system computes aggregate statistics for predicted candidate items based on the results sets from multiple different trained computational models. The machine learning system provides an interface for querying the result sets to access data representing predicted candidate items, including the aggregate statistics computed for the predicted candidate items. The aggregate statistics allow sorting, filtering, or otherwise prioritizing the predicted candidate items.

In one aspect, a process for machine learning includes training computational models using data representing a subset of researched items and applying the trained computational models to data representing a subset of potential candidate items to provide result sets for the computational models. A result set includes data representative of a set of predicted candidate items from among the potential candidate items. A computational model outputs, for each predicted candidate item, respective predicted information for a property of the predicted candidate item. The process includes computing aggregate statistics for predicted candidate items based on the results sets from multiple different trained computational models. The process includes querying the result sets to access data representing predicted candidate items, including the aggregate statistics computed for the predicted candidate items. The aggregate statistics allow sorting, filtering, or otherwise prioritizing the predicted candidate items.

In one aspect, a computer program product includes computer storage on which computer program instructions are stored. The computer program instructions configure a processing system to implement a machine learning system. The machine learning system trains computational models using data representing a subset of researched items and applies the trained computational models to data representing a subset of potential candidate items to provide result sets for the computational models. A result set includes data representative of a set of predicted candidate items from among the potential candidate items. A computational model outputs, for each predicted candidate item, respective predicted information for a property of the predicted candidate item. The machine learning system computes aggregate statistics for predicted candidate items based on the results sets from multiple different trained computational models. The machine learning system provides an interface for querying the result sets to access data representing predicted candidate items, including the aggregate statistics computed for the predicted candidate items. The aggregate statistics allow sorting, filtering, or otherwise prioritizing the predicted candidate items.

In one aspect, a machine learning system includes means for training computational models using data representing a subset of researched items, and means for applying the trained computational models to data representing a subset of potential candidate items to provide result sets for the computational models. A result set includes data representative of a set of predicted candidate items from among the potential candidate items. A computational model outputs, for each predicted candidate item, respective predicted information for a property of the predicted candidate item. The machine learning system includes means for computing aggregate statistics for predicted candidate items based on the results sets from multiple different trained computational models. The machine learning system includes means for querying the result sets to access data representing predicted candidate items, including the aggregate statistics computed for the predicted candidate items. The aggregate statistics allow sorting, filtering, or otherwise prioritizing the predicted candidate items.

In one aspect, a computer system comprises a processing device and computer storage. The computer storage stores data representing researched items and data representing potential candidate items. The computer storage further includes computer program instructions that, when processed by the processing system, configure the computer system to process a plurality of machine learning experiments, each machine learning experiment specified by a model set specifying a respective a. computational model, b. selected subset of the researched items and c. a selected subset of the potential candidate items. The computer program instructions configure the computer system to, for a model set, i. train the respective computational model using the data representing the respective selected subset of the researched items from the database, ii. apply the trained computational model for the model set to the data representing the selected subset the potential candidate items to generate and store in the database a respective result set for the model set, iii. compute aggregate statistics for predicted candidate items based on the results sets from the plurality of machine learning experiments, and iv. provide an interface for querying the result sets generated for the plurality of machine learning experiments to access data representing predicted candidate items, the accessed data including the aggregate statistics for the predicted candidate items. The result set of a model set comprises data representative of a set of predicted candidate items from among the potential candidate items, wherein the computational model outputs, based on the selected subset of researched items, for each predicted candidate item, respective predicted information for a property of the item.

In one aspect, a system stores result sets from the trained computational models as applied to data representing potential candidate items. A result set includes data representative of a set of predicted candidate items from among the potential candidate items. A computational model outputs, for each predicted candidate item, respective predicted information for a property of the predicted candidate item. The system computes aggregate statistics for predicted candidate items based on the results sets from multiple different trained computational models. The system provides an interface for querying the result sets to access data representing predicted candidate items, including the aggregate statistics computed for the predicted candidate items. The aggregate statistics allow sorting, filtering, or otherwise prioritizing the predicted candidate items.

In any of the foregoing, computer storage can include a database including: i. data representing a plurality of researched items and, for each researched item, respective information for a property of the item is known, wherein the property is among a plurality of types of properties, ii. data representing a plurality of potential candidate items wherein, for each potential candidate item, respective information is not known for at least one property among the plurality of types of properties, and iii. a plurality of model sets specifying a plurality of machine learning experiments.

In any of the foregoing, computer storage can include a database including a plurality of model sets specifying a plurality of machine learning experiments.

In any of the foregoing, a model set can specify a. a computational model, b. a selected subset of the plurality of researched items, and c. a selected subset of the plurality of potential candidate items.

In any of the foregoing, a researched item has respective information characterizing a selected type of property of the item among a plurality of types of property, and a potential candidate item does not have information for the selected type of property.

In any of the foregoing, an item can be a compound and a type of property can be a type of bioactivity in response to presence of the compound in or on a living thing.

In any of the foregoing, an interface can be provided to receive, for a model set, data representing a respective computational model, a respective selected subset of the plurality of research items, and a respective selected subset of the plurality of potential candidate items, and storing the model set in the database.

In any of the foregoing, the data describing how outputs of the multiple models in the ensemble are combined comprises data based on weights used by an ensemble function to combine outputs of the multiple models.

In any of the foregoing, the data based on weights comprises a first score generated without using the weights and a second score generated using the weights.

In any of the foregoing, the prediction value for a predicted candidate compound comprises a weighted conservative probability for bioactivity.

In any of the foregoing, the prediction value for a predicted candidate compound comprises a protein- or target-adjusted weighted conservative probability for bioactivity.

In any of the foregoing, the uncertainty model can be in itself a computational model that outputs the uncertainty value. A variety of kinds of model can be used. For example, the uncertainty model can be a Gaussian process model. The uncertainty model can be a self-supervision model. The uncertainty model can be a deep ensemble model. Input features for the uncertainty model can be derived in several ways, such as one or more of the following techniques. For example, the input features can be generated using various embedding techniques, such as autoencoders or other transforms, based on the data about the items processed by the primary model. The input features may include the output predictions of the primary model. The input features can include all or a subset of the input features of the primary model.

In any of the foregoing, the distance metric or similarity metric can be based on a distance metric or similarity metric over molecular fingerprints. The distance metric can be based on a Tanimoto distance over molecular fingerprints.

In any of the foregoing, the distance metric or similarity metric can be based on a distance metric or similarity metric over molecular features. The molecular features can be unreduced. The molecular features can be reduced. The molecular features can be reduced based on a PCA projection of molecular descriptor features. The distance metric can be based on a Euclidean distance.

In any of the foregoing, the computed statistics comprises a statistic based on a function of a distance metric or similarity metric between a predicted candidate item and its N nearest neighbors in the researched items used in the training dataset.

The foregoing description provides example implementations of a computer system implementing these techniques. The various computers used in this computer system can be implemented using one or more general-purpose computers, such as client devices including mobile devices and client computers, one or more server computers, or one or more database computers, or combinations of any two or more of these, which can be programmed to implement the functionality such as described in the example implementations.

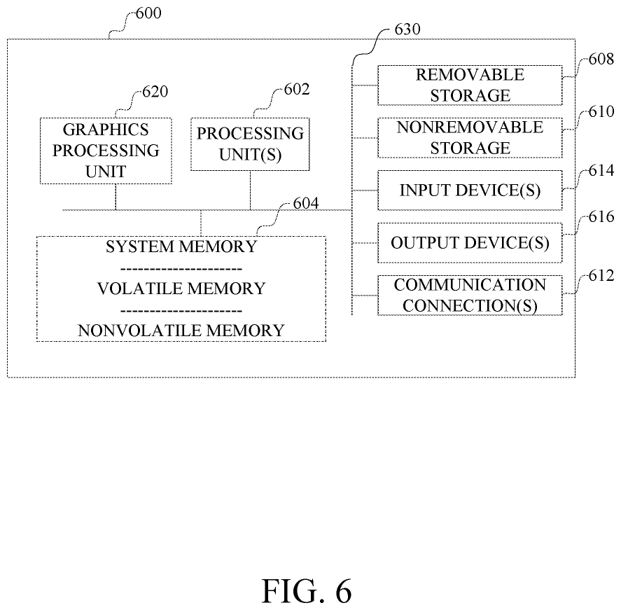

FIG. 6 is a block diagram of a general-purpose computer which processes computer programs using a processing system. Computer programs on a general-purpose computer generally include an operating system and applications. The operating system is a computer program running on the computer that manages access to resources of the computer by the applications and the operating system. The resources generally include memory, storage, communication interfaces, input devices and output devices.

Examples of such general-purpose computers include, but are not limited to, larger computer systems such as server computers, database computers, desktop computers, laptop, and notebook computers, as well as mobile or handheld computing devices, such as a tablet computer, handheld computer, smart phone, media player, personal data assistant, audio and/or video recorder, or wearable computing device.

With reference to FIG. 6, an example computer 600 comprises a processing system including at least one processing unit 602 and a memory 604. The computer can have multiple processing units 602 and multiple devices implementing the memory 604. A processing unit 602 can include one or more processing cores (not shown) that operate independently of each other. Additional co-processing units, such as graphics processing unit 620, also can be present in the computer. The memory 604 may include volatile devices (such as dynamic random-access memory (DRAM) or other random-access memory device), and non-volatile devices (such as a read-only memory, flash memory, and the like) or some combination of the two, and optionally including any memory available in a processing device. Other memory such as dedicated memory or registers also can reside in a processing unit. Such a memory configures is delineated by the dashed line 604 in FIG. 6. The computer 600 may include additional storage (removable and/or non-removable) including, but not limited to, solid state devices, or magnetically recorded or optically recorded disks or tape. Such additional storage is illustrated in FIG. 6 by removable storage 608 and non-removable storage 610. The various components in FIG. 6 are generally interconnected by an interconnection mechanism, such as one or more buses 630.

A computer storage medium is any medium in which data can be stored in and retrieved from addressable physical storage locations by the computer. Computer storage media includes volatile and nonvolatile memory devices, and removable and non-removable storage devices. Memory 604, removable storage 608 and non-removable storage 610 are all examples of computer storage media. Some examples of computer storage media are RAM, ROM, EEPROM, flash memory or other memory technology, CD-ROM, digital versatile disks (DVD) or other optically or magneto-optically recorded storage device, magnetic cassettes, magnetic tape, magnetic disk storage or other magnetic storage devices. Computer storage media and communication media are mutually exclusive categories of media.

The computer 600 may also include communications connection(s) 612 that allow the computer to communicate with other devices over a communication medium. Communication media typically transmit computer program code, data structures, program modules or other data over a wired or wireless substance by propagating a modulated data signal such as a carrier wave or other transport mechanism over the substance. The term “modulated data signal” means a signal that has one or more of its characteristics set or changed in such a manner as to encode information in the signal, thereby changing the configuration or state of the receiving device of the signal. By way of example, and not limitation, communication media includes wired media such as a wired network or direct-wired connection, and wireless media include any non-wired communication media that allows propagation of signals, such as acoustic, electromagnetic, electrical, optical, infrared, radio frequency and other signals. Communications connections 612 are devices, such as a network interface or radio transmitter, that interface with the communication media to transmit data over and receive data from signals propagated through communication media.

The communications connections can include one or more radio transmitters for telephonic communications over cellular telephone networks, and/or a wireless communication interface for wireless connection to a computer network. For example, a cellular connection, a Wi-Fi connection, a Bluetooth connection, and other connections may be present in the computer. Such connections support communication with other devices, such as to support voice or data communications.

The computer 600 may have various input device(s) 614 such as a various pointer (whether single pointer or multi-pointer) devices, such as a mouse, tablet and pen, touchpad and other touch-based input devices, stylus, image input devices, such as still and motion cameras, audio input devices, such as a microphone. The compute may have various output device(s) 616 such as a display, speakers, printers, and so on, also may be included. These devices are well known in the art and need not be discussed at length here.

The various storage 610, communication connections 612, output devices 616 and input devices 614 can be integrated within a housing of the computer, or can be connected through various input/output interface devices on the computer, in which case the reference numbers 610, 612, 614 and 616 can indicate either the interface for connection to a device or the device itself as the case may be.

An operating system of the computer typically includes computer programs, commonly called drivers, which manage access to the various storage 610, communication connections 612, output devices 616 and input devices 614. Such access generally includes managing inputs from and outputs to these devices. In the case of communication connections, the operating system also may include one or more computer programs for implementing communication protocols used to communicate information between computers and devices through the communication connections 612.

Any of the foregoing aspects may be embodied as a computer system, as any individual component of such a computer system, as a process performed by such a computer system or any individual component of such a computer system, or as an article of manufacture including computer storage in which computer program code is stored and which, when processed by the processing system(s) of one or more computers, configures the processing system(s) of the one or more computers to provide such a computer system or individual component of such a computer system.

Each component (which also may be called a “module” or “engine” or “computational model” or the like), of a computer system such as described herein, and which operates on one or more computers, can be implemented as computer program code processed by the processing system(s) of one or more computers. Computer program code includes computer-executable instructions and/or computer-interpreted instructions, such as program modules, which instructions are processed by a processing system of a computer. Generally, such instructions define routines, programs, objects, components, data structures, and so on, that, when processed by a processing system, instruct the processing system to perform operations on data or configure the processor or computer to implement various components or data structures in computer storage. A data structure is defined in a computer program and specifies how data is organized in computer storage, such as in a memory device or a storage device, so that the data can accessed, manipulated, and stored by a processing system of a computer.

Each reference, e.g., non-patent publications, patents, and patent applications, cited herein is hereby expressly incorporated by reference herein in its entirety. In the event of conflict between subject matter herein and subject matter in such a reference, the subject matter herein controls.

It should be understood that the subject matter defined in the appended claims is not necessarily limited to the specific implementations described above. The specific implementations described above are disclosed as examples only.

What is claimed is:

1. A machine learning system comprising:
a processing system configured to:
train computational models using data representing a subset of researched items; and
apply the trained computational models to data representing a subset of potential candidate items to provide result sets for the computational models,
wherein a result set includes data representative of a set of predicted candidate items from among the potential candidate items,
wherein a computational model outputs, for each predicted candidate item, respective predicted information for a property of the predicted candidate item, wherein the processing system further is configured to:
compute aggregate statistics for predicted candidate items based on the result sets from multiple different trained computational models; and
provide an interface for querying the result sets to access data representing predicted candidate items, including the aggregate statistics computed for the predicted candidate items, wherein the aggregate statistics allow sorting, filtering, or otherwise prioritizing the predicted candidate items.

2. The machine learning system in claim 1, wherein at least one computational model includes an ensemble of models.

3. The machine learning system in claim 2, wherein computed statistics includes data describing how outputs of the multiple models in the ensemble are combined.

4. The machine learning system in claim 1, wherein at least one computational model includes a primary model and an uncertainty model.

5. The machine learning system in claim 4, wherein the machine learning system is configured to:
train the primary model and the uncertainty model; and
apply the trained primary model and the trained uncertainty model to data representing potential candidate items;
wherein, for a predicted candidate item, the trained uncertainty model provides an uncertainty value for the item.

6. The machine learning system in claim 5, wherein the machine learning system is configured to compute statistics for prioritizing items based on the uncertainty values for items.

7. The machine learning system in claim 1, wherein the machine learning system is configured to compute weights for the selected subset of the plurality of researched items based on a distance metric or similarity metric between the researched items and the selected subset of potential candidate items.

8. The machine learning system in claim 7, wherein the computational model is trained using the weighted data representing the plurality of researched items.

9. The machine learning system in claim 8, wherein the computed statistics for the predicted candidate items are based on the distance metric or similarity metric between the researched items and the predicted candidate items.

10. The machine learning system in claim 1, wherein an item is a compound, the system further comprising:
selecting a predicted candidate compound predicted to have a type of bioactivity;
performing a laboratory experiment using the predicted candidate compound to obtain a quantitative measurement of the type of bioactivity in response to the selected predicted candidate compound; and
storing the quantitative measurement in the database of researched compounds.

11. The machine learning system in claim 10, wherein the researched compounds are small molecules.

12. The machine learning system in claim 10, wherein the researched compounds are drugs.

13. The machine learning system in claim 10, wherein the potential candidate compounds are proteins found in food.

14. The machine learning system in claim 10, wherein the potential candidate compounds are compounds found in food.

15. The machine learning system in claim 10, wherein the potential candidate compounds are compounds that are generally recognized as safe for human consumption.

16. The machine learning system in claim 10, wherein the potential candidate compounds are large naturally occurring molecules.

17. The machine learning system in claim 10, wherein the information characterizing bioactivity for a compound in the plurality of research compounds comprises measured and quantified bioactivity related to a protein in response to presence of the compound in a living thing.

18. The machine learning system in claim 10, wherein the selected type of bioactivity for a model set comprises bioactivity related to a selected protein in response to presence of a compound in a living thing, and wherein the selected subset of the plurality of researched compounds comprises researched compounds having information characterizing bioactivity related to the selected protein.

19. The machine learning system in claim 10, wherein the bioactivity related to a protein comprises bioactivity related to a concentration of the protein present in a living thing.

20. The machine learning system in claim 10, wherein the selected type of bioactivity is bioactivity related to a health condition of a living thing.

21. The machine learning system in claim 20, wherein the bioactivity related to a health condition is related to a concentration of protein present in the living thing.

22. The machine learning system in claim 10, further comprising an input interface that receives information characterizing verified bioactivity in response to presence of a selected one of the predicted candidate compounds, and that stores, in the database, data representing the selected one of the predicted candidate compounds as a researched compound among the plurality of researched compounds along with the respective information characterizing the verified bioactivity in response to presence of the selected one of the predicted candidate compounds.

23. The machine learning system in claim 17, wherein the living thing comprises plants.

24. The machine learning system in claim 17, wherein the living thing comprises animals.

25. The machine learning system in claim 24, wherein the living thing comprises mammals.

26. The machine learning system in claim 25, wherein the living thing comprises humans.

27. The machine learning system in claim 22, wherein the information characterizing bioactivity comprises a measured concentration of a protein in response to presence of a measured amount of a compound.

28. The machine learning system in claim 27, wherein the information is an amount in a continuous or semi-continuous range that indicates a concentration of an item in a sample.

29. The machine learning system in claim 28, wherein the information comprises a concentration of another item related to the amount of protein present in a sample.

30. The machine learning system in claim 27, wherein the candidate compounds are predicted to interact directly with the respective selected protein.

31. The machine learning system in claim 27, wherein the candidate compounds are predicted to interact indirectly with the respective selected protein.

32. The machine learning system in claim 27, wherein the candidate compounds are predicted to interact positively with the respective selected protein.

33. The machine learning system in claim 27, wherein the candidate compounds are predicted to interact negatively with the respective selected protein.

34. The machine learning system in claim 27, wherein the candidate compounds are predicted to interact independently with the respective selected protein.

35. The machine learning system in claim 27, wherein the candidate compounds are predicted to interact, when present with another compound, with the respective selected protein.

36. The machine learning system in claim 1, wherein querying includes identifying items that interfere with activity of a drug.

37. The machine learning system in claim 1, wherein querying includes identifying foods containing items that interfere with activity of a drug.

38. The machine learning system in claim 1, wherein querying includes identifying items that enhance activity of a drug.

39. The machine learning system in claim 1, wherein querying includes identifying foods containing items that enhance activity of a drug.

40. The machine learning system in claim 1, wherein querying includes aggregating interaction information for a plurality of items to characterize an overall effect of the plurality of items with respect to a health condition.

41. The machine learning system in claim 1, wherein querying includes aggregating interaction information for a plurality of items to characterize an overall effect of the plurality of items with respect to a drug.

42. The machine learning system in claim 17, further comprising performing assays with a candidate compound and the selected protein to characterize interaction of the candidate compound with the selected protein.

43. A machine learning system, comprising:
means for training computational models using data representing a subset of researched items;
means for applying the trained computational models to data representing a subset of potential candidate items to provide result sets for the computational models, wherein a result set includes data representative of a set of predicted candidate items from among the potential candidate items, and wherein a computational model outputs, for each predicted candidate item, respective predicted information for a property of the predicted candidate item;
means for computing aggregate statistics for predicted candidate items based on the results sets from multiple different trained computational models; and
means for querying the result sets to access data representing predicted candidate items, including the aggregate statistics computed for the predicted candidate items, wherein the aggregate statistics allow sorting, filtering, or otherwise prioritizing the predicted candidate items.

44. A computer system, comprising:
computer storage storing result sets from trained computational models as applied to data representing potential candidate items, wherein a result set includes data representative of a set of predicted candidate items from among the potential candidate items, wherein a computational model outputs, for each predicted candidate item, respective predicted information for a property of the predicted candidate item;
a processing system programmed to compute aggregate statistics for predicted candidate items based on the results sets from multiple different trained computational models; and
a user interface for querying the result sets to access data representing predicted candidate items, including the aggregate statistics computed for the predicted candidate items, wherein the aggregate statistics allow sorting, filtering, or otherwise prioritizing the predicted candidate items.
